SEQUENCE LISTING (8 sequences)

SEQ ID NO: 1 - DNA, 18 bp, Artificial Sequence
  PCR Primer
gaagaaggcggcgttgtc

SEQ ID NO: 2 - DNA, 19 bp, Artificial Sequence
  PCR Primer
gaggaaggtggaggtcagg

SEQ ID NO: 3 - DNA, 20 bp, Artificial Sequence
  PCR Primer
gctctctgctcctcctgttc

SEQ ID NO: 4 - DNA, 20 bp, Artificial Sequence
  PCR Primer
gactccgaccttcaccttcc

SEQ ID NO: 5 - protein, 168 aa, Homo sapiens
APEAQVSVQPNFQQDKFLGRWFSAGLASNSSWLREKKAALSMCKSVVAPATDGGLNLTSTFLRKNQCETRTMLLQPAGSLGSYSYRSPHWGSTYSVSVVETDYDQYALLYSQGSKGPGEDFRMATLYSRTQTPRAELKEKFTAFCKAQGFTEDTIVFLPQTDKCMTEQ

SEQ ID NO: 6 - protein, 190 aa, Homo sapiens
MATHHTLWMGLALLGVLGDLQAAPEAQVSVQPNFQQDKFLGRWFSAGLASNSSWLREKKAALSMCKSVVAPATDGGLNLTSTFLRKNQCETRTMLLQPAGSLGSYSYRSPHWGSTYSVSVVETDYDQYALLYSQGSKGPGEDFRMATLYSRTQTPRAELKEKFTAFCKAQGFTEDTIVFLPQTDKCMTEQ

SEQ ID NO: 7 - DNA, 573 bp, Homo sapiens
atggctactcatcacacgctgtggatgggactggccctgctgggggtgctgggcgacctgcaggcagcaccggaggcccaggtctccgtgcagcccaacttccagcaggacaagttcctggggcgctggttcagcgcgggcctcgcctccaactcgagctggctccgggagaagaaggcggcgttgtccatgtgcaagtctgtggtggcccctgccacggatggtggcctcaacctgacctccaccttcctcaggaaaaaccagtgtgagacccgaaccatgctgctgcagcccgcggggtccctcggctcctacagctaccggagtccccactggggcagcacctactccgtgtcagtggtggagaccgactacgaccagtacgcgctgctgtacagccagggcagcaagggccctggcgaggacttccgcatggccaccctctacagccgaacccagacccccagggctgagttaaaggagaaattcaccgccttctgcaaggcccagggcttcacagaggataccattgtcttcctgccccaaaccgataagtgcatgacggaacaatag

SEQ ID NO: 8 - DNA, 837 bp, Homo sapiens
gctcctcctgcacacctccctcgctctcccacaccactggcaccaggccccggacacccgctctgctgcaggagaatggctactcatcacacgctgtggatgggactggccctgctgggggtgctgggcgacctgcaggcagcaccggaggcccaggtctccgtgcagcccaacttccagcaggacaagttcctggggcgctggttcagcgcgggcctcgcctccaactcgagctggctccgggagaagaaggcggcgttgtccatgtgcaagtctgtggtggcccctgccacggatggtggcctcaacctgacctccaccttcctcaggaaaaaccagtgtgagacccgaaccatgctgctgcagcccgcggggtccctcggctcctacagctaccggagtccccactggggcagcacctactccgtgtcagtggtggagaccgactacgaccagtacgcgctgctgtacagccagggcagcaagggccctggcgaggacttccgcatggccaccctctacagccgaacccagacccccagggctgagttaaaggagaaattcaccgccttctgcaaggcccagggcttcacagaggataccattgtcttcctgccccaaaccgataagtgcatgacggaacaataggactccccagggctgaagctgggatcccggccagccaggtgacccccacgctctggatgtctctgctctgttccttccccgagcccctgccccggctccccgccaaagcaaccctgcccactcaggcttcatcctgcacaataaactccggaagcaagtcagtaaaaaaaaaaaaaaaaaaaaaaaaaa

(19) United States
(12) Patent Application Publication
(10) Pub. No.: US 2011/0318308 A1
(43) Pub. Date: Dec. 29, 2011

(54) LIPOCALIN-TYPE PROSTAGLANDIN D2 SYNTHASE AS A BIOMARKER FOR LUNG CANCER PROGRESSION AND PROGNOSIS

(75) Inventor: Louis RAGOLIA, Mineola, NY (US)

(73) Assignee: WINTHROP-UNIVERSITY HOSPITAL, Mineola, NY (US)

(21) Appl. No.: 13/168,223

(22) Filed: Jun. 24, 2011

Related U.S. Application Data
(60) Provisional application No. 61/358,704, filed on Jun. 25, 2010.

(51) Int. Cl. A61K 48/00; A61P 35/00; G01N 33/53; A61K 35/76; A61K 31/4166; C12Q 1/68

(52) U.S. Cl. 424/93.2; 514/389; 435/6.12; 435/7.92; 514/44 R

(57) ABSTRACT

A PGD(2) receptor (DP) deficiency enhances tumor progression accompanied by abnormal vascular expansion. In tumors, angiogenic endothelial cells highly express DP receptor, and its deficiency accelerates vascular leakage and angiogenesis. Administration of a synthetic DP agonist, BW245C, markedly suppresses tumor growth as well as tumor hyperpermeability in WT mice, but not in DP-deficient mice. In a corneal angiogenesis assay and a modified Miles assay, host DP deficiency potentiates angiogenesis and vascular hyperpermeability under COX-2-active situation, whereas exogenous administration of BW245C strongly inhibits both angiogenic properties in WT mice. In an in vitro assay, BW245C does not affect endothelial migration and tube formation, processes that are necessary for angiogenesis; however, it strongly improves endothelial barrier function via an increase in intracellular cAMP production. PGD(2)/DP receptor is a newly identified regulator of tumor vascular permeability, indicating DP agonism can be exploited as a therapy for the treatment of cancer.

### CROSS REFERENCE TO RELATED APPLICATION

This application claims priority to U.S. Provisional Application Ser. No. 61/358,704, filed Jun. 25, 2010, the entire contents of each of which are incorporated by reference herein.

### BACKGROUND

1. Technical Field

The present application relates to the field of biomarkers, and more particularly to biomarkers for lung cancer.

2. Description of the Art

Lung cancer is the leading cause of cancer death in both men and women in the United States with an expected 5-year survival rate of 16%. Since conventional therapy provides only limited success, translational research designed to improve outcomes with this disease is critical. The goal is to develop more effective chemopreventive and chemotherapeutic agents for the prevention and treatment of lung cancer.

The importance of prostaglandins (PGs) in tumor progression has been realized for several years. Harris R E. Cyclooxygenase-2 (cox-2) blockade in the chemoprevention of cancers of the colon, breast, prostate, and lung. Inflammopharmacology 2009; 17:55-67; Wang D, Dubois R N. Prostaglandins and cancer. Gut 2006; 55:115-22. Cyclooxygenase-2 (COX-2) derived PGE₂ can promote tumor growth by binding its receptors and activating signaling pathways which control cell proliferation, migration, apoptosis, and angiogenesis. Wang D, Dubois R N, “Prostaglandins and cancer”, Gut 55:115-122 (2006); Murata T, Lin M I, Aritake K, Matsumoto S, Narumiya S, Ozaki H, Urade Y, Hori M, Sessa W C, “Role of prostaglandin D2 receptor DP as a suppressor of tumor hyperpermeability and angiogenesis in vivo”, Proc Natl Acad Sci USA. 2008 Dec. 16; 105(50):20009-14. The predominance of COX activity in cell lines derived from human non-small cell carcinomas of the lung suggest that prostanoid biosynthesis may be characteristic of tumor cells comprising certain histological subclasses of human non-small cell carcinomas of the lung, particularly adenocarcinoma, bronchioloalveolar cell carcinoma, large cell undifferentiated carcinoma, and possibly adenosquamous carcinoma. Hubbard W C, Alley M C, Gray G N, Green K C, McLemore T L, Boyd M R, “Evidence for prostanoid biosynthesis as a biochemical feature of certain subclasses of non-small cell carcinomas of the lung as determined in established cell lines derived from human lung tumors”, Cancer Res 49:826-832 (1989)[1]. Both epidemiological studies and clinical trials indicate that prolonged use of non-steroidal anti-inflammatory drugs (NSAIDs) are associated with a decreased incidence of certain malignancies, including lung cancer. Wall R J, Shyr Y, Smalley W, “Nonsteroidal anti-inflammatory drugs and lung cancer risk: a population-based case control study”, J Thorac Oncol 2:109-114 (2007). The initial excitement of using COX-2 inhibitors as practical chemopreventives was dampened, however, by the undesirable cardiovascular side effects observed after prolonged use. Rahme E, Nedjar H, “Risks and benefits of COX-2 inhibitors vs. non-selective NSAIDs: does their cardiovascular risk exceed their gastrointestinal benefit? A retrospective cohort study”, Rheumatology (Oxford) 46:435-438 (2007); Solomon S D, McMurray J J, Pfeffer M A, Wittes J, Fowler R, Finn P, Anderson W F, Zauber A, Hawk E, Bertagnolli M, “Cardiovascular risk associated with celecoxib in a clinical trial for colorectal adenoma prevention”, N Engl J Med 352:1071-1080 (2005).

See also, Sargent L M, Ensell M X, Ostvold A C, Baldwin K T, Kashon M L, Lowry D T, Senft J R, Jefferson A M, Johnson R C, Li Z, Tyson F L, Reynolds S H, “Chromosomal changes in high- and low-invasive mouse lung adenocarcinoma cell strains derived from early passage mouse lung adenocarcinoma cell strains”, Toxicol Appl Pharmacol. 15:233(1):81-91 (2008); Sargent L M, Senft J R, Lowry D T, Jefferson A M, Tyson F L, Malkinson A M, Coleman A E, Reynolds S H, “Specific chromosomal aberrations in mouse lung adenocarcinoma cell lines detected by spectral karyotyping: a comparison with human lung adenocarcinoma”, Cancer Res 62:1152-1157 (2002).

L-PGDS is unique member of the lipocalin superfamily of proteins acting as both a lipophilic ligand-binding protein facilitating the transport of retinoids, thyroids and bile pigments, and possessing enzymatic activity catalyzing the isomerization of PG H₂ into PGD₂[2]. Tanaka T, Urade Y, Kimura H, Eguchi N, Nishikawa A, Hayaishi O, “Lipocalin-type prostaglandin D synthase (beta-trace) is a newly recognized type of retinoid transporter”, J Biol Chem 272:15789-15795 (1997). Originally purified from the central nervous system, L-PGDS comprises about four percent of the total cerebrospinal fluid protein, and has typically been associated with the regulation of the sleep-wake cycle and sensitivity to tactile pain. Urade Y, Hayaishi O, “Prostaglandin D synthase: structure and function. Vitam Horm”, 58:89-120 (2000). Several recent findings also demonstrate that L-PGDS has important vascular functions. Eguchi Y, Eguchi N, Oda H, Seiki K, Kijima Y, Matsu-ura Y, Urade Y, Hayaishi O, “Expression of lipocalin-type prostaglandin D synthase (beta-trace) in human heart and its accumulation in the coronary circulation of angina patients”, Proc Natl Acad Sci USA 94:14689-14694 (1997); Inoue T, Takayanagi K, Morooka S, Uehara Y, Oda H, Seiki K, Nakajima H, Urade Y, “Serum prostaglandin D synthase level after coronary angioplasty may predict occurrence of restenosis”, Thromb Haemost 85:165-170 (2001); Hirawa N, Uehara Y, Ikeda T, Gomi T, Hamano K, Totsuka Y, Yamakado M, Takagi M, Eguchi N, Oda H, Seiki K, Nakajima H, Urade Y, “Urinary prostaglandin D synthase (beta-trace) excretion increases in the early stage of diabetes mellitus”, Nephron 87:321-327 (2001); Miwa Y, Takiuchi S, Kamide K, Yoshii M, Horio T, Tanaka C, Banno M, Miyata T, Sasaguri T, Kawano Y, “Identification of gene polymorphism in lipocalin-type prostaglandin D synthase and its association with carotid atherosclerosis in Japanese hypertensive patients”, Biochem Biophys Res Commun 322:428-433 (2004); Hirawa N, Uehara Y, Yamakado M, Toya Y, Gomi T, Ikeda T, Eguchi Y, Takagi M, Oda H, Seiki K, Urade Y, Umemura S, “Lipocalin-type prostaglandin d synthase in essential hypertension”, Hypertension 39:449-454 (2002); as well as implications to cancer. Eichele K, Ramer R, Hinz B. Decisive role of cyclooxygenase-2 and lipocalin-type prostaglandin D synthase in chemotherapeutics-induced apoptosis of human cervical carcinoma cells. Oncogene 2008; 27:3032-44; Kim J, Yang P, Suraokar M, Sabichi A L, Llansa N D, Mendoza G, et al. Suppression of prostate tumor cell growth by stromal cell prostaglandinDsynthase-derived products. Cancer Res 2005; 65:6189-98; Sauter E R, Ehya H, Babb J, Diamandis E, Daly M, Klein-Szanto A, et al. Biological markers of risk in nipple aspirate fluid are associated with residual cancer and tumour size. Br J Cancer 1999; 81:1222-7; Su B, Guan M, Zhao R, Lu Y. Expression of prostaglandin D synthase in ovarian cancer. Clin Chem Lab Med 2001; 39:1198-203; Borchert G H, Melegos D N, Yu H, Giai M, Roagna R, Ponzone R, et al. Quantification of pepsinogen C and prostaglandin D synthase in breast cyst fluid and their potential utility for cyst type classification. Clin Biochem 1999; 32:39-44; Rogers M S, Rohan R M, Birsner A E, D'Amato R J. Genetic loci that control vascular endothelial growth factor-induced angiogenesis. FASEB J 2003; Takeda K, Yokoyama S, Aburatani H, Masuda T, Han F, Yoshizawa M, et al. Lipocalin-type prostaglandin D synthase as a melanocyte marker regulated by MITF. Biochem Biophys Res Commun 2006; 339:1098-106; Malki S, Bibeau F, Notarnicola C, Rogues S, Berta P, Poulat F, et al. Expression and biological role of the prostaglandin D synthase/SOX9 pathway in human ovarian cancer cells. Cancer Lett 2007; 255:182-93; Sasaki H, Nishikata I, Shiraga T, Akamatsu E, Fukami T, Hidaka T, et al. Overexpression of a cell adhesion molecule, TSLC1, as a possible molecular marker for acute-type adult T-cell leukemia. Blood 2005; 105:1204-13; Fujimori K, Kadoyama K, Urade Y. Protein kinase C activates human lipocalintype prostaglandin D synthase gene expression through de-repression of notch-HES signaling and enhancement of AP-2 beta function in brain-derived TE671 cells. J Biol Chem 2005; 280:18452-61; Garcia-Fernandez L F, Iniguez M A, Eguchi N, Fresno M, Urade Y, Munoz A. Dexamethasone induces lipocalin-type prostaglandin D synthase gene expression in mouse neuronal cells. J Neurochem 2000; 75:460-70; Yamashima T, Sakuda K, Tohma Y, Yamashita J, Oda H, Irikura D, et al. Prostaglandin D synthase (beta-trace) in human arachnoid and meningioma cells: roles as a cell marker or in cerebrospinal fluid absorption, tumorigenesis, and calcification process. J Neurosci 1997; 17:2376-82; Kawashima M, Suzuki S O, Yamashima T, Fukui M, Iwaki T. Prostaglandin D synthase (beta-trace) in meningeal hemangiopericytoma. Mod Pathol 2001; 14:197-201; Wei T, Geiser A G, Qian H R, Su C, Helvering L M, Kulkarini N H, et al. DNAmicroarray data integration by ortholog gene analysis reveals potential molecular mechanisms of estrogen-dependent growth of human uterine fibroids. BMC Womens Health 2007; 7:5; Mannino D M, Braman S. The epidemiology and economics of chronic obstructive pulmonary disease. Proc Am Thorac Soc 2007; 4:502-6.

For example: L-PGDS and PGD₂ metabolites produced by normal prostate stromal cells inhibited tumor cell growth through a peroxisome proliferator-activated receptor gamma (PPARγ)-dependent mechanism potentially contributing to the indolence and long latency period of this disease. Kim J, Yang P, Suraokar M, Sabichi A L, Llansa N D, Mendoza G, Subbarayan V, Logothetis C J, Newman R A, Lippman S M, Menter D G, “Suppression of prostate tumor cell growth by stromal cell prostaglandin D synthase-derived products”, Cancer Res 65:6189-6198 (2005). L-PGDS in nipple aspirate fluid is used to predict residual ductal carcinoma in situ (DCIS) or invasive cancer after needle or excisional biopsy of the breast. Sauter E R, Ehya H, Babb J, Diamandis E, Daly M, Klein-Szanto A, Sigurdson E, Hoffman J, Malick J, Engstrom P F, “Biological markers of risk in nipple aspirate fluid are associated with residual cancer and tumour size”, Br J Cancer 81:1222-1227 (1999); Nakamura M, Yamaguchi S, Motoyoshi K, Negishi M, Saito-Taki T, Matsumoto K, Hayashi I, Majima M, Kitasato H, “Anti-tumor effects of prostaglandin D2 and its metabolites, 15-deoxy-Δ12, 14-PGD2, by peroxisome proliferator-activated receptor (PPAR) γ-dependent and -independent pathways.” Inflammation and Regeneration 31:2-189-195 (2011). Expression of L-PGDS mRNA exists in ovarian cancer, and is related to the cancer type. Su B, Guan M, Zhao R, Lu Y, “Expression of prostaglandin D synthase in ovarian cancer”, Clin Chem Lab Med 39:1198-1203 (2001). Quantification of L-PGDS in breast cyst fluid may be useful in the subclassification of cyst type in patients with gross cystic disease. Borchert G H, Melegos D N, Yu H, Giai M, Roagna R, Ponzone R, Sgro L, Diamandis E P, “Quantification of pepsinogen C and prostaglandin D synthase in breast cyst fluid and their potential utility for cyst type classification”, Clin Biochem 32:39-44 (1999); L-PGDS has been identified as a genetic loci controlling VEGF-induced angiogenesis, Rogers M S, Rohan R M, Birsner A E, D'Amato R J, “Genetic loci that control vascular endothelial growth factor-induced angiogenesis”, Faseb J (2003). L-PGDS mRNA is present in melanocytes but undetectable in human melanoma cell lines. Takeda K, Yokoyama S, Aburatani H, Masuda T, Han F, Yoshizawa M, Yamaki N, Yamamoto H, Eguchi N, Urade Y, Shibahara S, “Lipocalin-type prostaglandin D synthase as a melanocyte marker regulated by MITF”, Biochem Biophys Res Commun 339:1098-1106 (2006). L-PGDS may be a possible diagnostic marker for ovarian carcinomas. Malki S, Bibeau F, Notarnicola C, Rogues S, Berta P, Poulat F, Boizet-Bonhoure B, “Expression and biological role of the prostaglandin D synthase/SOX9 pathway in human ovarian cancer cells”, Cancer Lett 255:182-193 (2007). It is also a possible diagnostic marker for adult T-cell leukemia. Sasaki H, Nishikata I, Shiraga T, Akamatsu E, Fukami T, Hidaka T, Kubuki Y, Okayama A, Hamada K, Okabe H, Murakami Y, Tsubouchi H, Morishita K, “Overexpression of a cell adhesion molecule, TSLC1, as a possible molecular marker for acute-type adult T-cell leukemia”, Blood 105:1204-1213 (2005). A novel transcriptional regulatory mechanism is responsible for the high level expression of the human L-PGDS gene in TE671 (medulloblastoma of cerebellum) cells. Fujimori K, Kadoyama K, Urade Y, “Protein kinase C activates human lipocalin-type prostaglandin D synthase gene expression through de-repression of notch-HES signaling and enhancement of AP-2 beta function in brain-derived TE671 cells”, J Biol Chem 280:18452-18461 (2005). L-PGDS is differentially expressed in melanoma patients after vaccination with a tumor-specific antigen. Mannino D M, Braman S, “The epidemiology and economics of chronic obstructive pulmonary disease”, Proc Am Thorac Soc 4:502-506 (2007). The tumor promoter 12-O-tetradecanoyl-phorbol 13-acetate (TPA), which induces the synthesis of PGs in many tissues, inhibits L-PGDS expression. Garcia-Fernandez L F, Iniguez M A, Eguchi N, Fresno M, Urade Y, Munoz A, “Dexamethasone induces lipocalin-type prostaglandin D synthase gene expression in mouse neuronal cells”, J Neurochem 75:460-470 (2000). Functional differences in various types of meningeal cells are attributable to differences in L-PGDS expression with meningioma cells showing intense L-PGDS immunoreactivity in the perinuclear region. Yamashima T, Sakuda K, Tohma Y, Yamashita J, Oda H, Irikura D, Eguchi N, Beuckmann C T, Kanaoka Y, Urade Y, Hayaishi O, “Prostaglandin D synthase (beta-trace) in human arachnoid and meningioma cells: roles as a cell marker or in cerebrospinal fluid absorption, tumorigenesis, and calcification process”, J Neurosci 17:2376-2382 (1997); Kawashima M, Suzuki S O, Yamashima T, Fukui M, Iwaki T, “Prostaglandin D synthase (beta-trace) in meningeal hemangiopericytoma”, Mod Pathol 14:197-201 (2001); L-PGDS expression is related to estrogen-dependent cell survival and leiomyoma tumor growth. Wei T, Geiser A G, Qian H R, Su C, Helvering L M, Kulkarini N H, Shou J, N'Cho M, Bryant H U, Onyia J E, “DNA microarray data integration by ortholog gene analysis reveals potential molecular mechanisms of estrogen-dependent growth of human uterine fibroids”, BMC Womens Health 7:5 (2007). L-PGDS gene amplification represents a novel method of calibration for erythroblastic leukemia viral oncogene homolog 2 in breast cancer aiding prognosis. Mannino D M, Braman S, “The epidemiology and economics of chronic obstructive pulmonary disease”, Proc Am Thorac Soc 4:502-506 (2007); L-PGDS-mediated effects on cell proliferation, apoptosis and migration in various cell lines suggest a possible role in cancer progression. Ragolia L, Palaia T, Paric E, Maesaka J K. Prostaglandin D2 synthase inhibits the exaggerated growth phenotype of spontaneously hypertensive rat vascular smooth muscle cells. J Biol Chem 2003; 278:22175-81; Ragolia L, Palaia T, Koutrouby T B, Maesaka J K. Inhibition of cell cycle progression and migration of vascular smooth muscle cells by prostaglandin D2 synthase: resistance in diabetic Goto-Kakizaki rats. Am J Physiol Cell Physiol 2004; 287:C1273-81; Maesaka J K, Palaia T, Frese L, Fishbane S, Ragolia L. Prostaglandin D(2) synthase induces apoptosis in pig kidney LLC-PK1 cells. Kidney Int 2001; 60:1692-8.

The biochemical relationships with its precursor, arachadonic acid, and metabolits PGF_{2α}, PGE₂, PGD₂, and more distantly PGD₂, are shown in FIG. 8. For example, vascular smooth muscle cells isolated from diabetic rats as well as spontaneously hypertensive rats commonly display hyper-proliferative phenotypes. L-PGDS suppresses the exaggerated proliferation of cells isolated from hypertensive animals. Ragolia L, Palaia T, Paric E, Maesaka J K, “Prostaglandin D2 synthase inhibits the exaggerated growth phenotype of spontaneously hypertensive rat vascular smooth muscle cells”, J Biol Chem 278:22175-22181 (2003). It also suppresses excess proliferation of cells from diabetic animals. Ragolia L, Palaia T, Koutrouby T B, Maesaka J K, “Inhibition of cell cycle progression and migration of vascular smooth muscle cells by prostaglandin D2 synthase: resistance in diabetic Goto-Kakizaki rats”, Am J Physiol Cell Physiol 287:C1273-1281 (2004). Two complementary mechanisms are involed: i) the stimulation of apoptosis; and ii) the inhibition of cell proliferation by stalling cell cycle progression. L-PGDS-induced apoptosis was confirmed by the TUNEL assay, annexin V staining, electron microscopy, and caspase3 activity, and was both time and dose dependent. Data suggests that glycosylation alters the apoptotic potency of L-PGDS. Maesaka J K, Palaia T, Frese L, Fishbane S, Ragolia L, “Prostaglandin D(2) synthase induces apoptosis in pig kidney LLC-PK1 cells”, Kidney Int 60:1692-1698 (2001). Concomitant with its effect on apoptosis, L-PGDS was shown to inhibit excess cell proliferation by stalling cell cycle progression. Immunoblot analysis of cell cycle proteins clearly demonstrated the regulatory role of L-PGDS in cell cycle progression and the resistance observed in diabetic cells. Ragolia L, Palaia T, Koutrouby T B, Maesaka J K, “Inhibition of cell cycle progression and migration of vascular smooth muscle cells by prostaglandin D2 synthase: resistance in diabetic Goto-Kakizaki rats”, Am J Physiol Cell Physiol 287:C1273-1281 (2004). In the case of cyclin D1 and cdk2, L-PGDS was able to inhibit serum-induced protein expression in wildtype cells, but failed to do so in diabetic cells. There were no L-PGDS effects on either cyclin D3 or p27^{Kip1} protein expression, although there were alterations of their gene expressions in wildtype cells. In addition, serum-induced protein expression of p21^{Cip1} was inhibited by L-PGDS in wildtype cells and not diabetic cells, implicating this cyclin-dependent kinase inhibitor. Finally, L-PGDS inhibits PDGF-induced VSMC migration in control VSMCs but not diabetic cells. Ragolia L, Palaia T, Koutrouby T B, Maesaka J K, “Inhibition of cell cycle progression and migration of vascular smooth muscle cells by prostaglandin D2 synthase: resistance in diabetic Goto-Kakizaki rats”, Am J Physiol Cell Physiol 287:C1273-1281 (2004).

PKC is a family of serine/threonine kinases traditionally associated with the regulation of cell proliferation and differentiation. Nishizuka Y, “The molecular heterogeneity of protein kinase C and its implications for cellular regulation”, Nature 334:661-665 (1988). Certain isoforms are linked to the induction of apoptosis. Powell C T, Brittis N J, Stec D, Hug H, Heston W D, Fair W R, “Persistent membrane translocation of protein kinase C alpha during 12-0-tetradecanoylphorbol-13-acetate-induced apoptosis of LNCaP human prostate cancer cells”, Cell Growth Differ 7:419-428 (1996); Day M L, Zhao X, Wu S, Swanson P E, Humphrey P A, “Phorbol ester-induced apoptosis is accompanied by NGFI-A and c-fos activation in androgen-sensitive prostate cancer cells”, Cell Growth Differ 5:735-741 (1994). PKCs have been linked to carcinogenesis since PKC activators can act as tumor promoters. Furthermore, functional studies have suggested that PKCs play a role in the carcinogenesis and maintenance of malignant phenotype. PMA-induced apoptosis is mediated by L-PGDS phosphorylation and is accompanied by the inhibition of the phosphatidylinositol 3-kinase (PI3-K) and protein kinase B (Akt) pathways. Ragolia L, Palaia T, Paric E, Maesaka J K, “Elevated L-PGDS activity contributes to PMA-induced apoptosis concomitant with downregulation of PI3-K”, Am J Physiol Cell Physiol 284:C119-126 (2003). Akt, GSK-3β, and Rb phosphorylations are inhibited by L-PGDS. In addition, sustained MAPK activity, via a reduction of MKP-1, accompanies the increased insulin-stimulated cell proliferation observed in hypertensive cells. Begum N, Ragolia L, Rienzie J, McCarthy M, Duddy N, “Regulation of mitogen-activated protein kinase phosphatase-1 induction by insulin in vascular smooth muscle cells. Evaluation of the role of the nitric oxide signaling pathway and potential defects in hypertension”, J Biol Chem 273:25164-25170 (1998), Basal MKP-2 expression is elevated in L-PGDS KO's, and the addition of exogenous L-PGDS abolishes MKP-2 expression. Ragolia L, Palaia T, Hall C E, Maesaka J K, Eguchi N, Urade Y, “Accelerated glucose intolerance, nephropathy, and atherosclerosis in prostaglandin D2 synthase knock-out mice”, J Biol Chem 280:29946-29955 (2005). Under conditions of PKC stimulation such as inflammation there is increased PKC activation leading to increased L-PGDS serine phosphorylation which results in the hypo-phosphorylation and activation of Bad, as well as the hypo-phosphorylation of retinoblastoma (pRb), both signaling increased apoptosis, See FIG. 9. The ability to induce apoptosis with phorbol ester vanished in a cell line with depleted L-PGDS protein expression.

Preliminary data indicate that PKC and p38MAPK signaling play a significant role in L-PGDS-mediated effects. The molecular mechanisms and signaling pathways responsible for L-PGDS action using a combination of PG's, synthetic DP1/DP2 receptor ligands, and specific pathway inhibitors in cultured A549 cells may be identified. L-PGDS may work autonomously or via the production of PGD₂, and if so the role of the DP1 and DP2 receptors, or if one of the downstream PGD₂ derivatives working through PPARγ may be involved. Specific inhibitors of PI3-K, Akt, and PKC signaling, all pathways which have previously been determined to have a role in L-PGDS-induced apoptosis, may be utilized to tease out the signaling mechanisms responsible for L-PGDS action.

PGD₂, the enzymatic product of L-PGDS, and its metabolites also have interesting links with cancer. For example: PGD₂ has been linked to the inhibition of ovarian cancer. Miyauchi M, Kikuchi Y, Kizawa I, Oomori K, Kita T, Kato K, “[Inhibition of human ovarian cancer cell growth by prostaglandin D2]”, Nippon Sanka Fujinka Gakkai Zasshi 39:215-220 (1987); Kikuchi Y, Miyauchi M, Oomori K, Kita T, Kizawa I, Kato K, “Inhibition of human ovarian cancer cell growth in vitro and in nude mice by prostaglandin D2”, Cancer Res 46:3364-3366 (1986). Human erythromyeloblastoid leukemia cell proliferation is inhibited by PGD₂. Santoro M G, Crisari A, Benedetto A, Amici C, “Modulation of the growth of a human erythroleukemic cell line (K562) by prostaglandins: antiproliferative action of prostaglandin A”, Cancer Res 46:6073-6077 (1986). PGD₂ has been suggested to represent a rational target for therapies aimed at reducing the incidence of colitis-associated colorectal cancer. Zamuner S R, Bak A W, Devchand P R, Wallace J L, “Predisposition to colorectal cancer in rats with resolved colitis: role of cyclooxygenase-2-derived prostaglandin d2”, Am J Pathol 167:1293-1300(2005). PGD₂ induces various transduction pathways and activates the function of SOX9. Malki S, Declosmenil F, Farhat A, Moniot B, Poulat F, Boizet-Bonhoure B, “[Prostaglandin D2: new roles in the embryonic and pathological gonad]”, Med Sci (Paris) 24:177-183 (2008). SOX9 is a transcription factor of which methylation has been linked to lung cancer. Cortese R, Hartmann O, Berlin K, Eckhardt F, “Correlative gene expression and DNA methylation profiling in lung development nominate new biomarkers in lung cancer”, Int J Biochem Cell Biol 40:1494-1508 (2008). Additionally, PGD₂ is non-enzymatically converted to 15-deoxy Δ^{12,14} PGD₂ (15d-PGJ₂), a natural ligand for PPARγ, which plays an important role in the death of malignant T lymphocytes (Jurkat cells). Ferreira-Silva V, Rodrigues A C, Hirata T D, Hirabara S M, Curi R, “Effects of 15-deoxy-Delta12, 14 prostaglandin J2 and ciglitazone on human cancer cell cycle progression and death: the role of PPARgamma”, Eur J Pharmacol 580:80-86 (2008). In addition, PPARγ, expressed in lung cancer cells, can be activated by various ligands and can inhibit lung cancer cell growth through the induction of apoptosis. Zhang M, Zou P, Bai M, Jin Y, Tao X, “Peroxisome proliferator-activated receptor-gamma activated by ligands can inhibit human lung cancer cell growth through induction of apoptosis”, J Huazhong Univ Sci Technolog Med Sci 23:138-140 (2003). 15d-PGD₂ inhibits growth of A549 and H460 non-small-cell lung cancer cell lines and xenograft tumors; and in lung tumor cells, 15d-PGD₂ enhances the anti-tumor action of docetaxel by PPARγ-dependent and -independent mechanisms mediated by the induction of apoptosis. Fulzele S V, Chatterjee A, Shaik M S, Jackson T, Ichite N, Singh M, “15-Deoxy-Delta12,14-prostaglandin J2 enhances docetaxel anti-tumor activity against A549 and H460 non-small-cell lung cancer cell lines and xenograft tumors”, Anticancer Drugs 18:65-78 (2007). Furthermore, growth suppression of PPARγ expressing tumor cells by PGD₂ metabolites in the prostate microenvironment is likely to be an endogenous mechanism involved in tumor suppression that potentially contributes to the indolence and long latency period of this disease. Kim J, Yang P, Suraokar M, Sabichi A L, Llansa N D, Mendoza G, Subbarayan V, Logothetis C J, Newman R A, Lippman S M, Menter D G, “Suppression of prostate tumor cell growth by stromal cell prostaglandin D synthase-derived products”, Cancer Res 65:6189-6198 (2005).

PGs can enhance or suppress inflammation in response to tumor growth by acting on various receptors. Two G protein-coupled receptors for PGD₂, DP1 and DP2 have been identified. Activation of DP1 leads to the stimulation of adenylate cyclase activity and increased intracellular cAMP levels. DP2, is preferentially expressed on T-helper (Th) 2-type cells, T-cytotoxic (Tc) 2 cells, eosinophils and basophils. Tsuda H, Michimata T, Sakai M, Nagata K, Nakamura M, Saito S, “A novel surface molecule of Th2- and Tc2-type cells, CRTH2 expression on human peripheral and decidual CD4+ and CD8+ T cells during the early stage of pregnancy”, Clin Exp Immunol 123:105-111 (2001). DP2 induces intracellular calcium mobilization and chemotaxis in a Gαi-dependent manner. Hirai H, Tanaka K, Yoshie O, Ogawa K, Kenmotsu K, Takamori Y, Ichimasa M, Sugamura K, Nakamura M, Takano S, Nagata K, “Prostaglandin D2 selectively induces chemotaxis in T helper type 2 cells, eosinophils, and basophils via seven-transmembrane receptor CRTH2”, J Exp Med 193:255-261 (2001). One current set of data suggests that PGD₂ participates in the immunologic mechanisms which serve to establish and maintain pregnancy. Successful pregnancy implies avoidance of rejection of paternal antigens of fetal tissues by the maternal immune system. An important mechanism behind this immunological paradox involves the down-regulation of the cellular immune response. T-helper (Th) 1 and T-cytotoxic (Tc) 1 cells which produce interleukin (IL)-2, interferon (IFN)-γ, and tumour necrosis factor (TNF)-β are suppressed while Th2 and Tc2 cells, which produce IL-4, IL-6, IL-10 and IL-13 are upregulated. Michimata T, Ogasawara M S, Tsuda H, Suzumori K, Aoki K, Sakai M, Fujimura M, Nagata K, Nakamura M, Saito S, “Distributions of endometrial NK cells, B cells, T cells, and Th2/Tc2 cells fail to predict pregnancy outcome following recurrent abortion”, In: Am J Reprod Immunol; 196-202 (2002; Michimata T, Tsuda H, Sakai M, Fujimura M, Nagata K, Nakamura M, Saito S, “Accumulation of CRTH2-positive T-helper 2 and T-cytotoxic 2 cells at implantation sites of human decidua in a prostaglandin D(2)-mediated manner”, Mol Hum Reprod 8:181-187 (2002); Saito S, Tsuda H, Michimata T, “Prostaglandin D2 and reproduction”, Am J Reprod Immunol 47:295-302 (2002); Wegmann T G, Lin H, Guilbert L, Mosmann T R, “Bidirectional cytokine interactions in the maternal-fetal relationship: is successful pregnancy a TH2 phenomenon?”, Immunol Today 14:353-356 (1993). Present studies indicate possible imbalanced expression of prostanoid receptors in colorectal cancer compared to normal colon tissue without clear cut relationship to disease progression. Gustafsson A, Hansson E, Kressner U, Nordgren S, Andersson M, Lonnroth C, Lundholm K, “Prostanoid receptor expression in colorectal cancer related to tumor stage, differentiation and progression”, Acta Oncol 46:1107-1112 (2007).

### SUMMARY

Prostaglandin D₂ (PGD₂) is a mediator in various pathophysiological processes, including inflammation and tumorigenesis. PGD₂ can be converted to active metabolites and is known to activate two distinct receptors, DP and chemoattractant receptor-homologous molecule expressed on Th2 cells (CRTH2/DP2). In the past, PGD₂ was thought to be involved only in the process of inflammation. However, in recent years, several studies have shown that PGD₂ has anti-proliferative ability against tumorigenesis and can induce cellular apoptosis via activation of the caspase-dependent pathway in human colorectal cancer cells, leukemia cells and eosinophils. In the lung, where PGD₂ is highly released when sensitized mast cells are challenged with allergen, the mechanism of PGD₂-induced apoptosis is unclear.

A549 cells, a type of non-small cell lung carcinoma (NSCLC) were treated with PGD₂ under various conditions, including while blocking DP and CRTH2/DP2 with the selective antagonists BWA868C and ramatroban, respectively. PGD₂ induces A549 cell death through the intrinsic apoptotic pathway, although the process does not appear to involve either DP or CRTH2/DP2. Similar results were also found with H2199 cells, other type of NSCLC. PGD₂ metabolites induce apoptosis effectively and that 15d-PGD2 is a likely candidate for the principal apoptotic inducer in PGD₂-induced apoptosis in non-small cell lung carcinoma A549 cells.

Altered dynamic expression of PGD₂ and its metabolites, via genetic L-PGDS modulation, is believed to alter susceptibility to carcinogen-induced lung cancer in mice. PGD₂ receptors, DP 1 and DP2, represent new therapeutic intervention points in the treatment of lung cancer. Some of the signaling pathways involved in L-PGDS action as well as the mechanisms through which L-PGDS regulates the delicate balance of PGs and cytokines during tumor progression are identified, and thus available as diagnostic and therapeutic targets. L-PGDS phosphorylation likely plays a role in relation to tumor progression, by reducing apoptosis.

While it appears that broad inhibition of PG synthesis is excessively simple (see FIG. 8), one aspect of the present invention provides regulation at L-PGDS, a point downstream of PGH₂, which may provide the mechanism necessary for fine-tuning PG signaling in a temporal and tissue-specific manner and offer a more efficacious chemotherapeutic site.

Lipocalin-type prostaglandin D₂ synthase (L-PGDS) inhibits the progression of the cell cycle and also induces apoptosis in multiple cell lines. Furthermore, significantly less L-PGDS gene and protein expression is demonstrated in human non-small cell lung cancer (NSCLC) tumor types as compared to normal margins.

Lipocalin-type prostaglandin D₂ synthase (L-PGDS) induces apoptosis and prevents cell cycle progression in several cell types. The expression of L-PGDS in a variety of human lung tumor types has been demonstrated. While L-PGDS expression was evident in the surrounding margins, significantly decreased protein and gene expression was observed in the tumor tissue. Using RTPCR, L-PGDS gene expression was shown to be decreased proportionately with tumor progression. In addition, exogenously added L-PGDS suppresses the hyperproliferation and PDGF-stimulated migration of A549 cells, a cultured carcinomic human alveolar basal epithelial cell line. L-PGDS may play a key role in modulating lung cancer growth and may offer a novel diagnostic and therapeutic approach for treatment. Ragolia L, Palaia T, Paric E, Maesaka J K, “Prostaglandin D2 synthase inhibits the exaggerated growth phenotype of spontaneously hypertensive rat vascular smooth muscle cells”, J Biol Chem 278:22175-22181 (2003).

The expression of L-PGDS in lung tumors and the surrounding margin was examined. L-PGDS gene expression in lung tumors was monitored at various stages of progression using quantitative RT-PCR and the effects of exogenously added L-PGDS on proliferation and PDGF-stimulated migration of A549 cells, a cultured carcinomic human alveolar basal epithelial cell line, was investigated.

Accordingly, the present invention provides a method for, diagnosing, detecting treating and predicting a future disease progression, predicting a responsiveness to pharmacological agents, predicting a metastatic state, and/or staging, a non small cell lung cancer (NSCLC), based on the differences in lipocalin-type prostaglandin D₂ synthase (L-PGDS) metabolism and expression activity between normal cells and various stages of cancer.

NSCLC can be detected or diagnosed by ascertaining anomalous expression patterns of L-PGDS in tissue, and in particular, differences may be with respect to a normal margin, or other control tissue.

NSCLC can be treated by disrupting the L-PGDS physiology of the malignant tissue, to induce apoptosis of the cells or prevent proliferation and metastasis. This can be achieved by administering pharmacological agents and/or gene therapy to the organism or particular tissues. The gene administered may, for example, encode Lipocalin-type prostaglandin D₂ synthase (EC=5.3.99.2), e.g., SEQ ID NO:005

```
                10 20 30 40 50 60
MATHHTLWMG LALLGVLGDL QAAPEAQVSV QPNFQQDKFL GRWFSAGLAS NSSWLREKKA
                70 80 90 100 110 120
ALSMCKSVVA PATDGGLNLT STFLRKNQCE TRTMLLQPAG SLGSYSYRSP HWGSTYSVSV
              130 140 150 160 170 180
VETDYDQYAL LYSQGSKGPG EDFRMATLYS RTQTPRAELK EKFTAFCKAQ GFTEDTIVFL
              190
PQTDKCMTEQ
```

(www.uniprot.org/uniprot/P41222, Homo sapiens, expressly incorporated herein by reference).

Alternately, animal forms of the protein and/or synthetic or modified sequences may be employed. For example, residue may be changed from Arginine to Glutamine (R56Q), see expasy.org/cgi-bin/variant_pages/get-sprot-variant.pl?VAR_{—}004273. The therapeutic sequence may truncate the signal peptide residues 1-22, and use only the mature form residues 23-190, SEQ ID NO: 006:

APEAQVSVQP NFQQDKFLGR WFSAGLASNS SWLREKKAAL
SMCKSVVAPA TDGGLNLTST FLRKNQCETR TMLLQPAGSL
GSYSYRSPHW GSTYSVSVVE TDYDQYALLY SQGSKGPGED
FRMATLYSRT QTPRAELKEK FTAFCKAQGF TEDTIVFLPQ
TDKCMTEQ

The Lipocalin-type Prostaglandin D₂ Synthase of Homo sapiens is at LOCUS NM_{—}000954, and is transcribed as a 837 bp linear mRNA. The L-PGDS isolated from brain is 21 kDa. L-PGDS is variously known as beta-trace protein; PGD2 synthase; lipocalin-type prostaglandin D synthase; glutathione-independent PGD synthase; cerebrin-28; prostaglandin-D2 synthase; glutathione-independent PGD synthetase; lipocalin-type prostaglandin-D synthase, PTGDS; L-PGDS; LPGDS; PDS; PGD2; PGDS; PGDS2.

The gene as the sequence SEQ ID NO:007:

| | |
|---|---|
| 1 | gctcctcctg cacacctccc tcgctctccc acaccactgg caccaggccc cggacacccg |
| 61 | ctctgctgca ggagaatggc tactcatcac acgctgtgga tgggactggc cctgctgggg |
| 121 | gtgctgggcg acctgcaggc agcaccggag gcccaggtct ccgtgcagcc caacttccag |
| 181 | caggacaagt tcctggggcg ctggttcagc gcgggcctcg cctccaactc gagctggctc |
| 241 | cgggagaaga aggcggcgtt gtccatgtgc aagtctgtgg tggcccctgc cacggatggt |
| 301 | ggcctcaacc tgacctccac cttcctcagg aaaaaccagt gtgagacccg aaccatgctg |
| 361 | ctgcagcccg cggggtccct cggctcctac agctaccgga gtccccactg gggcagcacc |
| 421 | tactccgtgt cagtggtgga gaccgactac gaccagtacg cgctgctgta cagccagggc |
| 481 | agcaagggcc ctggcgagga cttccgcatg gccaccctct acagccgaac ccagaccccc |
| 541 | agggctgagt taaaggagaa attcaccgcc ttctgcaagg cccagggctt cacagaggat |
| 601 | accattgtct tcctgcccca aaccgataag tgcatgacgg aacaatagga ctccccaggg |
| 661 | ctgaagctgg gatcccggcc agccaggtga cccccacgct ctggatgtct ctgctctgtt |
| 721 | ccttccccga gcccctgccc cggctccccg ccaaagcaac cctgcccact caggcttcat |
| 781 | cctgcacaat aaactccgga agcaagtcag taaaaaaaaa aaaaaaaaaa aaaaaaa |

The coding sequence is as follows SEQ ID NO:008:

| | |
|---|---|
| 1 | atggctactc atcacacgct gtggatggga ctggccctgc tgggggtgct gggcgacctg |
| 61 | caggcagcac cggaggccca ggtctccgtg cagcccaact tccagcagga caagttcctg |
| 121 | gggcgctggt tcagcgcggg cctcgcctcc aactcgagct ggctccggga gaagaaggcg |
| 181 | gcgttgtcca tgtgcaagtc tgtggtggcc cctgccacgg atggtggcct caacctgacc |
| 241 | tccaccttcc tcaggaaaaa ccagtgtgag acccgaacca tgctgctgca gcccgcgggg |
| 301 | tccctcggct cctacagcta ccggagtccc cactggggca gcacctactc cgtgtcagtg |
| 361 | gtggagaccg actacgacca gtacgcgctg ctgtacagcc agggcagcaa gggccctggc |
| 421 | gaggacttcc gcatggccac cctctacagc cgaacccaga cccccagggc tgagttaaag |
| 481 | gagaaattca ccgccttctg caaggcccag ggcttcacag aggataccat tgtcttcctg |
| 541 | ccccaaaccg ataagtgcat gacggaacaa tag |

The sequence may of course be modified in known manner, for example to increase or reduce enzymatic activity:

59 K→A: Increases enzyme activity about two-fold.

64 M→A: Reduces enzyme activity almost ten-fold.

79 L→A: Reduces enzyme activity over ten-fold.

83 F→A: Reduces enzyme activity about five-fold.

131 L→A: Reduces enzyme activity almost ten-fold.

149 Y→A: Increases enzyme activity about two-fold.

See, Zhou Y., Shaw N., Li Y., Zhao Y., Zhang R., Liu Z. J., “Structure-function analysis of human 1-prostaglandin D synthase bound with fatty acid molecules.”, FASEB J. 24:4668-4677(2010) [PubMed: 20667974], expressly incorporated herein by reference.

The DNA coding sequence may be as defined by: useast.ensembl.org/Homo_{—}sapiens/Transcript/Summary?g=ENSG00000107317;r=9:139871957-139876190;t=ENST00000371625, the entirety of which, and linked pages, are expressly incorporated herein by reference. See also, White, D M, Mikol D D, Espinosal R, Weimer B, Le Beau M M, Stefansson K, “Structure and Chromosomal Localization of the Human Gene for a Brain Form of Prostaglandin D2 Synthase”, The Journal of Biological Chemistry, 267, 23202-23208 (1992), expressly incorporated herein by reference.

The invention also provides methods for detecting the L-PGDS peptides, gene or mRNA in a test sample for use in diagnosing the presence, absence or progression of a disease, or for prognosing a likely future course of a disease with respect to absence of treatment or various available therapeutic interventions. The test sample includes but is not limited to a biological sample such as tissue, blood, serum or biological fluid.

The invention also provides a method for monitoring the disease progression and the treatment progress. The invention also provides a method for monitoring the disease progression and treatment regime, i.e., aggressive treatment proposed if prognosis is poor or is otherwise poor.

The present invention provides a method for treating lung, colon, and prostate (e.g., neoplastic) diseases, comprising: identifying a subject having lung, colon or prostate disease; and administering to a patient to one or more compositions that increase PGD₂ levels, L-PGDS levels or activity or expression, in the lung, colon or prostate to alter a course of the subject having such disease. The subject may be human or animal. The composition is provided in a pharmaceutically acceptable carrier, in an effective dose. The composition is provided in an amount that avoids substantial toxicity to the subject while achieving an efficacious treatment.

It is therefore an object to provide a method of treating a non small cell lung cancer, comprising administering an effective amount of a Prostaglandin D₂ (PGD₂) receptor agonist, in a pharmaceutically acceptable form, to a patient having non small cell lung cancer, in sufficient quantity to treat the non small cell lung cancer. The PGD₂ receptor agonist may, for example, comprise at least one of BW245C and BW868C.

The method may further comprise performing an assay on the non small cell lung cancer cells to determine a Lipocalin-type prostaglandin D2 synthase (L-PGDS) activity or expression of the tissue, and administering the PGD2 agonist selectively in dependence on a determined low level of L-PGDS in the non small cell lung cancer cells.

Another object provides a method of diagnosing, staging or predicting outcome of a non small cell lung cancer tumor, comprising testing cells of the non small cell lung cancer tumor for at least one of indicia or mRNA level corresponding to the Lipocalin-type prostaglandin D synthase (L-PGDS) gene, L-DPGS gene product, and PGD2 level, and scoring the test result with respect to non-cancer lung cells. The at least one indicia or mRNA level corresponding to the Lipocalin-type prostaglandin D synthase (L-PGDS) gene, L-PGDS gene product, and PGD2 level of the cells if showing at least a 25% reduction as compared to non-cancer lung cells from the same patient, may indicate a cancerous or precancerous condition. For example, a 40% reduction threshold may be employed to indicate a threshold for treatment. A 60% reduction as compared to non-cancer lung cells from the same patient may indicate, for example, a stage Ia cancer; an 80% reduction as compared to non-cancer lung cells from the same patient may indicate, for example, a stage a stage Ib cancer; a 90% reduction as compared to non-cancer lung cells from the same patient may indicate, for example, a stage a stage II cancer, and a 95% reduction as compared to non-cancer lung cells from the same patient may indicate, for example, a stage a stage IV cancer. For example, L-PGDS activity can be measured by RT-PCR using primers designed to specifically amplify L-PGDS mRNA. An mRNA level of Lipocalin-type prostaglandin D synthase (L-PGDS) gene transcript of the tested cells of less than 40% of non-cancerous lung cells from the same patient may indicate a poor prognosis if the patient is left untreated. An mRNA level of Lipocalin-type prostaglandin D synthase (L-PGDS) gene transcript of the tested cells of less than 40% of non-cancerous lung cells from the same patient may also be interpreted to indicate a likely effective response of the non small cell lung cancer tumor to a therapy which increases L-PGDS activity or agonizes PGD2 receptors in the non small cell lung cancer tumor.

A further object provides a method of treating a non small cell lung cancer tumor in a patient, comprising testing cells from a biopsy of the non small cell lung cancer tumor for at least one of indicia or mRNA corresponding to the Lipocalin-type prostaglandin D synthase (L-PGDS) gene, L-PGDS gene product, and PGD2 level, and comparing the biopsied non small cell lung cancer tumor cells with control lung cells, and treating the non small cell lung cancer tumor with a treatment to increase L-PGDS or agonize PGD2 receptors in the non small cell lung cancer tumor selectively in dependence on the testing, wherein a reduced level of the at least one of indicia or mRNA corresponding to the Lipocalin-type prostaglandin D synthase (L-PGDS) gene, L-PGDS gene product, and PGD2 level indicates a likely favorable response to the treatment.

A still further object provides a method of treating a patient having a non small cell lung cancer, comprising administering an effective amount of a gene therapy configured to cause expression in lung tissue of the patient of Lipocalin-type prostaglandin D synthase (L-PGDS). The gene therapy may, for example, comprises a genetically engineered adenovirus or SV40 virus comprising DNA encoding an L-PGDS. The L-PGDS may comprise a human L-PGDS EC=5.3.99.2, a mutant or synthetic form having higher or lower enzymatic activity, or a non-human enzyme. For example, the expressed L-PGDS may have an activity in the lung tissue higher than normal human L-PGDS. The gene therapy may be applied intratracheally.

Combination therapies are specifically contemplated. Thus, in addition to administering an enzyme or a gene with encodes an enzymatically active produce, a patient may also receive a drug with serves as a substrate for the enzyme, or interacts with the same receptor as an enzyme product. Thus, for example, in addition to gene therapy for inducing increased L-PGDS activity in the patient's tumor, a PGD₂ receptor agonist may be administered to the patient. The PGD₂ receptor agonist, for example, may comprise at least one of BW245C and BW868C.

It is also an object to provide a method to predict pathological characteristics of a non small cell lung cancer tumor in a patient, comprising: performing an assay to determine expression of a gene encoding an L-PGDS in the non small cell lung cancer tumor and a non-tumor margin; and categorizing the pathological characteristics of the non small cell lung cancer tumor, selectively in dependence on the assay. The patient may be further treated in accordance with the categorized pathological characteristics.

### BRIEF DESCRIPTION OF THE FIGURES

FIGS. 1A-1E show significantly less L-PGDS protein expression in non-small cell lung cancer (NSCLC) as compared to normal margins;

FIG. 2 shows that L-PGDS protein expression, as determined by fluorescence L-PGDs staining in the tumor tissue, decreases with adenocarcinoma stage.

FIG. 3A shows a Western blot insert of L-PGDS protein expression in tumor versus margin;

FIG. 3B shows a graph which demonstrated that L-PGDs gene expression is decreased several-fold in tumor when compared to normal tissue;

FIG. 4 shows a graph which indicates that L-PGDS gene expression decreases with tumor progression, demonstrating that L-PGDs gene expression decreased proportionally with the stage of tumor progression when compared to normal lung tissue;

FIG. 5A shows Western blots of L-PGDS protein expression in a lung carcinoma cell line versus control;

FIG. 5B shows a graph indicating that L-PGDS expression is lower in A549 lung carcinoma cells than in controls;

FIG. 6 shows a graph which indicates that exogenous L-PGDS suppresses A549 hyperproliferation;

FIG. 7 shows micrographs which reveal that exogenous L-PGDS inhibits A549 migration;

FIG. 8 shows a biochemical pathway for arachadonic acid metabolism;

FIG. 9 shows a physiological pathway for the relationship of phorbol esters and inflammation on apoptosis;

FIG. 10 shows a photograph of stained mouse lung showing lungs from control, L-PGDS knockouts, and transgenic L-PGDS overexpressing mice, two weeks after lung tumor induction.

### DESCRIPTION

Development of more effective chemopreventive and chemotherapeutic agents with minimal toxicity to treat lung cancer is crucial. L-PGDS represents a very attractive site for the prevention/treatment of lung cancer for several reasons. First, L-PGDS induces cellular apoptosis, delays cell cycle progression, and inhibits cell proliferation and migration in multiple cell types. These are all important processes involved in tumor progression. In addition, L-PGDS, PGD₂ and its metabolites have also been linked to lung cancer. Secondly, recent efforts have attempted to illustrate the importance of identifying the molecular mechanisms by which PGE₂ promotes tumor growth and metastasis in order to develop safer strategies for cancer prevention and treatment. The balance between L-PGDS and PGE synthase, has incidentally been described as a major determinant of other disease processes such as atherosclerosis. Cipollone F, Fazia M, Iezzi A, Ciabattoni G, Pini B, Cuccurullo C, Ucchino S, Spigonardo F, De Luca M, Prontera C, Chiarelli F, Cuccurullo F, Mezzetti A, “Balance between PGD synthase and PGE synthase is a major determinant of atherosclerotic plaque instability in humans”, Arterioscler Thromb Vasc Biol 24:1259-1265 (2004). It is believed that the balance between L-PGDS and PGE synthase is also significant to lung cancer progression. Finally, an inverse relationship has been shown between L-PGDS gene and protein expression and lung tumor progression. Specific DP1/DP2 agonists and antagonists are available and equally attractive to study as potential therapeutics, e.g., BW245C (5-(6-carboxyhexyl)-1-(3-cyclohexyl-3-hydroxypropyl-hydantoin), AS702224, TS-022, 15R-methyl-PGD₂, 13-14-dihydro-15-keto-PGD₂, AM156, AM206, L-745870, 15R-PGD(2), MK-0524, BWA868C, BW24-SC, BAY-u3405, 15-deoxy-Delta12,14-prostaglandin J2 (15d-PGD2), 11-deoxy-11-methylene PGD₂, G₀6983 (PKCα,Δ,ε,ζ), G₀6976 (PKCα), GF10923X (PKCα,Δ,ε), LY333531 (PKC β), SB203580 (p38MAPK), SB203580, CD200, FGF18, GPRC5D, GPR49, LRRC15, Serpin A, CDT6, BMP2, LHX2, THBS1, MYCN, NR4A2, MEST, TM4SF1, CRLF1, TNFRSF12A, SELENBP1, GPR161, HEPH, FZD7, and CLIC4, CCL18, Col11A1, Col3A1, CD4, Cd1a, FCER1A, HLA-C, HLA-DPA1, IGF1, GPR105, PDGFRL, ADRA2A, CCL19, CORN, 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxo-cyclopent-2-enyl)hept-5-enoate, (Z)—N-ethyl-7-((R)-2-4R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopen-t-2-enyl)hept-5-enamide, (Z)-7-((R)-2-((R,E)-3-hydroxy-4-(3-(trifluoromethyl)phenoxy)but-1-enyl)-5- -oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclope-nt-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. These agents may be used alone or in combination, and may be administered concurrently or sequentially. See also, 2011/0144160, 2011/0130453, 2011/0112134, 2011/0098352, 2011/0098302, 2011/0071175, 2011/0060026, 2011/0034558, 2011/0028717, 2011/0021599, 2011/0021573, 2011/0002866, 2010/0330077, each of which is expressly incorporated herein by reference.

Pharmaceutical compositions for use in accordance with the present invention may be formulated in conventional manner using one or more physiologically acceptable carriers or excipients.

A “pharmaceutically acceptable salt” is any salt that retains the activity of the parent compound and does not impart any additional deleterious or untoward effects on the subject to which it is administered and in the context in which it is administered compared to the parent compound.

Pharmaceutically acceptable salts of acidic functional groups may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted, or has different toxicology, bioavailability or pharmacology profile.

Thus, the compounds and their physiologically acceptable salts and solvates may be formulated for administration by inhalation or insufflation (either through the mouth or the nose) or oral, buccal, parenteral, rectal, bronchial or topical administration.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent. Preparations for oral administration may be suitably formulated to give controlled release of the active compound.

For solid dosage forms, non-toxic solid carriers include, but are not limited to, pharmaceutical grades of mannitol, lactose, starch, magnesium stearate, sodium saccharin, the polyalkylene glycols, talcum, cellulose, glucose, sucrose and magnesium carbonate. The solid dosage forms may be uncoated or they may be coated by known techniques to delay disintegration and absorption in the gastrointestinal tract and thereby provide a sustained action over a longer period. For example, a time delay material such as glyceryl monostearate or glyceryl distcarate may be employed. They may also be coated by the technique described in the U.S. Pat. Nos. 4,256,108; 4,166,452; and 4,265,874 to form osmotic therapeutic tablets for control release. Liquid pharmaceutically administrable dosage forms can, for example, comprise a solution or suspension of one or more of the presently useful compounds and optional pharmaceutical adjutants in a carrier, such as for example, water, saline, aqueous dextrose, glycerol, ethanol and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980. The composition of the formulation to be administered, in any event, contains a quantity of one or more of the presently useful compounds in an amount effective to provide the desired therapeutic effect.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Compounds may be used for the treatment of the respiratory tract by nasal, bronchial or buccal administration of, for example, aerosols or sprays which can disperse the pharmacological active ingredient in the form of a powder or in the form of drops of a solution or suspension. Pharmaceutical compositions with powder-dispersing properties usually contain, in addition to the active ingredient, a liquid propellant with a boiling point below room temperature and, if desired, adjuncts, such as liquid or solid non-ionic or anionic surfactants and/or diluents. Pharmaceutical compositions in which the pharmacological active ingredient is in solution contain, in addition to this, a suitable propellant, and furthermore, if necessary, an additional solvent and/or a stabiliser. Instead of the propellant, compressed air can also be used, it being possible for this to be produced as required by means of a suitable compression and expansion device.

A number of medicinal aerosol formulations using propellant systems are disclosed in, for example, U.S. Pat. No. 6,613,307 and the references cited therein (such as, for example, EP 0372777, WO91/04011, WO91/11173. WO91/11495, WO91/14422, WO92/00107, WO93/08447, WO93/08446. WO93/11743, WO93/11744 and WO93/11745) all of which are incorporated by reference herein in their entirety. The propellants for use in the invention may be any fluorocarbon, hydrogen-containing fluorocarbon or hydrogen-containing chlorofluorocarbon propellant or mixtures thereof having a sufficient vapour pressure to render them effective as propellants. The propellant may additionally contain a volatile adjuvant such as a saturated hydrocarbon for example propane, n-butane, isobutane, pentane and isopentane or a dialkyl ether for example dimethyl ether.

Where a surfactant is employed in the aerosol, it is selected from those which are physiologically acceptable upon administration by inhalation such as oleic acid, sorbitan trioleate (Span R 85), sorbitan mono-oleate, sorbitan monolaurate, polyoxyethylene (20) sorbitan monolaurate, polyoxyethylene (20) sorbitan monooleate, natural lecithin, fluorinated and perfluorinated surfactants including fluorinated lecithins, fluorinated phosphatidylcholines, oleyl polyoxyethylene (2) ether, stearyl polyoxyethylene (2) ether, lauryl polyoxyethylene (4) ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil and sunflower seed oil. See, for example, U.S. Pat. No. 6,613,307.

The compounds may be formulated for parenteral administration by injection, either subcutaneously, intramuscularly or intravenously, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol and the like. In addition, if desired, the injectable pharmaceutical compositions to be administered may also contain minor amounts of non-toxic auxiliary substances such as wetting or emulsifying agents, pH buffering agents and the like. Parenteral formulations will generally be sterile.

The compounds may also be formulated in rectal compositions such as suppositories or retention enemas, e.g., containing conventional suppository bases such as cocoa butter or other glycerides.

For topical use, creams, ointments, gels, solutions or suspensions, etc., containing the compound disclosed herein are employed. Topical formulations may generally be comprised of a pharmaceutical carrier, cosolvent, emulsifier, penetration enhancer, preservative system, and emollient.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Those skilled in the art will readily understand that for administration the compounds disclosed herein can be admixed with pharmaceutically acceptable excipients which per se are well known in the art. Specifically, a drug to be administered systemically, it may be confected as a powder, pill, tablet or the like, or as a solution, emulsion, suspension, aerosol, syrup or elixir suitable for oral or parenteral administration or inhalation.

The amount of the presently useful compound or compounds administered is, of course, dependent on the therapeutic effect or effects desired, on the specific mammal being treated, on the severity and nature of the mammal's condition, on the manner of administration, on the potency and pharmacodynamics of the particular compound or compounds employed, and on the judgement of the prescribing physician.

Preservatives that may be used in the pharmaceutical compositions of the present invention include, but are not limited to, benzalkonium chloride, chlorobutanol, thimerosal, phenylmercuric acetate and phenylmercuric nitrate. A useful surfactant is, for example, Tween 80. Likewise, various useful vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose and purified water. See, www.pharmainfo.net/reviews/analysis-preservatives-pharmaceutical-products.

The carrier, or, if more than one be present, each of the carriers, must be acceptable in the sense of being compatible with the other ingredients of the formulation and not deleterious to the recipient.

Typically, the dose of a prostaglandin agonist or antagonist will be about 0.01 to 100 mg/kg; so as to maintain the concentration of drug in the plasma at a concentration effective to agonize or antagonize the receptor. The precise amount of a compound which is therapeutically effective, and the route by which such compound is best administered, is readily determined by one of ordinary skill in the art by comparing the blood level of the agent to the concentration required to have a therapeutic effect. The actual dose of the active compounds of the present invention depends on the specific compound, and on the condition to be treated; the selection of the appropriate dose is well within the knowledge of the skilled artisan.

Tonicity adjustors may be added as needed or convenient. They include, but are not limited to, salts, particularly sodium chloride, potassium chloride, mannitol and glycerin, or any other suitable ophthalmically acceptable tonicity adjustor.

Other excipient components which may be included in the ophthalmic preparations are chelating agents. A useful chelating agent is edentate disodium, although other chelating agents may also be used in place or in conjunction with it.

See, e.g., U.S. 2002/0022218, 2004/0162323, 2011/0142855, 2004/0122059, each of which expressly incorporated herein by reference.

L-PGDS, or other relevant peptides disclosed herein, can be delivered intra-tracheally to the lung, or for absorption into the vascular system. See, Patton J S, Fishburn C S, and Weers J G, Komada F, Iwakawa S, Yamamoto N, Sakakibara H, Okumura K, “The Lungs as a Portal of Entry for Systemic Drug Delivery”, Proceedings of the American Thoracic Society 1:338-344 (2004); “Intratracheal delivery of peptide and protein agents: Absorption from solution and dry powder by rat lung”, Journal of Pharmaceutical Sciences, 83(6):863-867 (1994); Ghilzai N K, “Pulmonary Drug Delivery”, www.drugdel.com/Pulm_review.pdf; Pietropaolo M, Olson C D, Reiseter B S, Kasaian M T, Happ M P, “Intratracheal administration to the lung enhances therapeutic benefit of an MBP peptide in the treatment of murine experimental autoimmune encephalomyelitis”, Clin Immunol. 2000 May; 95(2):104-16; Andrade F; Videira M; Ferreira D; Sarmento B, “Nanocarriers for Pulmonary Administration of Peptides and Therapeutic Proteins”, Nanomedicine. 2011; 6(1):123-141; Umashankar M, Sachdeva, Gulati M: Aquasomes: a promising carrier for peptides and protein delivery. Nanomedicine6, 419-426 (2010); Klingler C, Muller B, Steckel H: Insulin-micro- and nanoparticles for pulmonary delivery. Int. J. Pharm. 377(1-2), 173-179 (2009); Amidi M, Mastrobattista E, Jiskoot W, Hennink W: Chitosan-based delivery systems for protein therapeutics and antigens. Adv. Drug Deliv. Rev. 62(1), 59-82 (2010); Sarmento B, Ribeiro A, Veiga F, Ferreira D: Development and characterization of new insulin containing polysaccharide nanoparticles. Colloids Surf. B Biointerfaces 53(2), 193-202 (2006); Bayat A, Dorkoosh F, Dehpour A et al.: Nanoparticles of quaternized chitosan derivatives as a carrier for colon delivery of insulin: ex vivo and in vivo studies. Int. J. Pharm. 356(1-2), 259-266 (2008); Bailey M, Berkland C: Nanoparticle formulations in pulmonary drug delivery. Med. Res. Rev. 29(1), 196-212 (2009); Nyambura B, Kellaway I, Taylor K: Insulin nanoparticles: stability and aerosolization from pressurized metered dose inhalers. Int. J. Pharm. 375(1-2), 114-122 (2009); Sung J, Pulliam B, Edwards D: Nanoparticles for drug delivery to the lungs. Trends Biotechnol. 25(12), 563-570 (2007); Soppimath K, Aminabhavi T, Kulkarni A, Rudzinski W: Biodegradable polymeric nanoparticles as drug delivery devices. J. Control. Release 70(1-2), 1-20 (2001); Martins S, Sarmento B, Ferreira D, Souto E: Lipid-based colloidal carriers for peptide and protein delivery-liposomes versus lipid nanoparticles. Int. J. Nanomedicine 2(4), 595-607 (2007); Tauzin B: Biotechnology Medicines in Development. Pharmaceutical Research and Manufacturers of America (PhRMA), Washington, D.C., USA (2008); EvaluatePharma®: World preview to 2016. EvaluatePharma Ltd, London, UK (2010); Tang L, Meibohm B: Pharmacokinetics of peptides and proteins. In: Pharmacokinetics and Pharmacodynamics of Biotech Drugs—Principles and Case Studies in Drug Development. Meibohm B (Ed.). Wiley-VCH Verlag GmbH & Co. KGaA, Weinheim, Germany (2006); Hillery A: Drug delivery: The basic concepts. In: Drug Delivery and Targeting for Pharmacists and Pharmaceutical Scientists. Hillery A, Lloyd A, Swarbrick J (Eds). Taylor & Francis, London, UK (2001); Morishita M, Peppas N: Is the oral route possible for peptide and protein drug delivery? Drug Discov. Today 11(19-20), 905-910 (2006); Antosova Z, Mackova M, Kral V, Macek T: Therapeutic application of peptides and proteins: parenteral forever? Trends Biotechnol. 27(11), 628-635 (2009); Cryan S: Carrier-based strategies for targeting protein and peptide drugs to the lungs. AAPS J. 7(1),E20-E41 (2005); Cryan S, Sivadas N, Garcia-Contreras L: In vivo animal models for drug delivery across the lung mucosal barrier. Adv. Drug Deliv. Rev. 59(11), 1133-1151 (2007); Iazzetti G, Rigutti E (Eds): Atlas of Anatomy. Taj Books, Surrey, UK (2005); Yang W, Peters J, Williams R R: Inhaled nanoparticles—a current review. Int. J. Pharm. 356(1-2), 239-247 (2008); Groneberg D, Fischer A, Chung K, Daniel H: Molecular mechanisms of pulmonary peptidomimetic drug and peptide transport. Am. J. Respir. Cell Mol. Biol. 30(3), 251-260 (2004); Grenha A, Carrión-Recio D, Teijeiro-Osorio D, Seijo B, Remuñán-López C (Eds): Nano and Microparticulate Carriers for Pulmonary Drug Delivery. American Scientific Publishers, Valencia, Calif., USA (2008); Tomoda K, Ohkoshi T, Nakajima T, Makino K: Preparation and properties of inhalable nanocomposite particles: effects of the size, weight ratio of the primary nanoparticles in nanocomposite particles and temperature at a spray-dryer inlet upon properties of nanocomposite particles. Colloids Surf. B Biointerfaces 64(1), 70-76 (2008); Smola M, Vandamme T, Sokolowski A: Nanocarriers as pulmonary drug delivery systems to treat and to diagnose respiratory and non respiratory diseases. Int. J. Nanomedicine 3(1), 1-19 (2008); Cefalu W: Concept, strategies, and feasibility of noninvasive insulin delivery. Diabetes Care 27(1), 239-246 (2004); Kim K, Malik A: Protein transport across the lung epithelial barrier. Am. J. Physiol Lung Cell. Mol. Physiol. 284(2),L247-L259 (2003); Hastings R, Folkesson H, Matthay M: Mechanisms of alveolar protein clearance in the intact lung. Am. J. Physiol. Lung Cell Mol. Physiol. 286(4),L679-L689 (2004); Berthiaume Y, Albertine K, Grady M, Fick G, Matthay M: Protein clearance from the air spaces and lungs of unanesthetized sheep over 144 h. J. Appl. Physiol. 67(5), 1887-1897 (1989); Agu R, Ugwoke M, Armand M, Kinget R, Verbeke N: The lung as a route for systemic delivery of therapeutic proteins and peptides. Respir. Res. 2(4), 198-209 (2001); Pezron I, Mitra R, Pal D, Mitra A: Insulin aggregation and asymmetric transport across human bronchial epithelial cell monolayers (calu-3). J. Pharm. Sci. 91(4), 1135-1146 (2002); Folkesson H, Weström B, Karlsson B: Permeability of the respiratory tract to different-sized macromolecules after intratracheal instillation in young and adult rats. Acta Physiol. Scand. 139(2), 347-354 (1990); Conhaim R, Watson K, Lai-Fook S, Harms B: Transport properties of alveolar epithelium measured by molecular hetastarch absorption in isolated rat lungs. J. Appl. Physiol. 91(4), 1730-1740 (2001); Holter J, Weiland J, Pacht E, Gadek J, Davis W: Protein permeability in the adult respiratory distress syndrome. Loss of size selectivity of the alveolar epithelium. J. Clin. Invest. 78(6), 1513-1522 (1986); Matsukawa Y, Yamahara H, Yamashita F, Lee V, Crandall E, Kim K: Rates of protein transport across rat alveolar epithelial cell monolayers. J. Drug Target 7(5), 335-342 (2000); Bahadduri P, D'souza V, Pinsonneault J et al.: Functional characterization of the peptide transporter pept2 in primary cultures of human upper airway epithelium. Am. J. Respir. Cell Mol. Biol. 32(4), 319-325 (2005); Islam N, Gladki E: Dry powder inhalers (DPIs)—a review of device reliability and innovation. Int. J. Pharm. 360(1-2), 1-11 (2008); Brunton L, Lazo J, Parker K (Eds): Goodman & Gilman's The Pharmacological Basis of therapeutics. McGraw-Hill Professional, Berkshire, UK (2006); Katzung B (Ed.): Basic & Clinical Pharmacology. Lange Medical Books/McGraw-Hill, New York, N.Y., USA (2001); Cheng K, Mahato R: Biopharmaceutical challenges: pulmonary delivery of proteins and peptides. In: Pharmacokinetics and Pharmacodynamics of Biotech Drugs—Principles and Case Studies in Drug Development, Meibohm B (Ed.): Wiley-VCH Verlag GmbH & Co. KGaA, Weinheim, Germany (2006); Wolzt M, De La Peña A, Berclaz P, Tibaldi F, Gates J, Muchmore D: Air inhaled insulin versus subcutaneous insulin: pharmacokinetics, glucodynamics, and pulmonary function in asthma. Diabetes Care 31(4), 735-740 (2008); De Galan B, Simsek S, Tack C, Heine R: Efficacy and safety of inhaled insulin in the treatment of diabetes mellitus. Neth. J. Med. 64(9), 319-325 (2006); Skyler J, Cefalu W, Kourides I et al.: Efficacy of inhaled human insulin in Type 1 diabetes mellitus: a randomised proof-of-concept study. Lancet 357(9253), 331-335 (2001); Rosenstock J, Cefalu W, Hollander P et al.: Two-year pulmonary safety and efficacy of inhaled human insulin (Exubera) in adult patients with Type 2 diabetes. Diabetes Care 31(9), 1723-1728 (2008); Karathanasis E, Bhavane R, Annapragada A: Glucose-sensing pulmonary delivery of human insulin to the systemic circulation of rats. Int. J. Nanomedicine 2(3), 501-513 (2007); Ungaro F, D'emmanuele Di Villa Bianca R, Giovino C et al.: Insulin-loaded plga/cyclodextrin large porous particles with improved aerosolization properties: in vivo deposition and hypoglycaemic activity after delivery to rat lungs. J. Control. Release 135(1), 25-34 (2009); Garg S, Kelly W: Insulin delivery via lungs—is it still possible? Diabetes Technol. Ther. 11(2 Suppl. 2),S1-S3 (2009); Peppas N, Carr D: Impact of absorption and transport on intelligent therapeutics and nano-scale delivery of protein therapeutic agents. Chem. Eng. Sci. 64(22), 4553-4565 (2009); Lee Y, Sinko P: Oral delivery of salmon calcitonin. Adv. Drug Deliv. Rev. 42(3), 225-238 (2000); Patton J: Pulmonary delivery of drugs for bone disorders. Adv. Drug Deliv. Rev. 42(3), 239-248 (2000); Deftos L, Nolan J, Seely B et al.: Intrapulmonary drug delivery of salmon calcitonin. Calcif. Tissue Int. 61(4), 345-347 (1997); Yamamoto A, Okumura S, Fukuda Y, Fukui M, Takahashi K, Muranishi S: Improvement of the pulmonary absorption of (asu1,7)-eel calcitonin by various absorption enhancers and their pulmonary toxicity in rats. J. Pharm. Sci. 86(10), 1144-1147 (1997); Waldrep J, Arppe J, Jansa K, Vidgren M: Experimental pulmonary delivery of cyclosporin a by liposome aerosol. Int. J. Pharm. 160,239-249 (1998); Mitruka S, Pham S, Zeevi A et al.: Aerosol cyclosporine prevents acute allograft rejection in experimental lung transplantation. J. Thorac. Cardiovasc. Surg. 115(1), 28-36, discussion 36-27 (1998); Keenan R, Iacono A, Dauber J et al.: Treatment of refractory acute allograft rejection with aerosolized cyclosporine in lung transplant recipients. J. Thorac. Cardiovasc. Surg. 113(2), 335-340, discussion 340-331 (1997); Letsou G, Safi H, Reardon M et al.: Pharmacokinetics of liposomal aerosolized cyclosporine a for pulmonary immunosuppression. Ann. Thorac. Surg. 68(6), 2044-2048 (1999); Thipphawong J: Inhaled cytokines and cytokine antagonists. Adv. Drug Deliv. Rev. 58(9-10), 1089-1105 (2006); Skubitz K, Anderson P: Inhalational interleukin-2 liposomes for pulmonary metastases: a Phase I clinical trial. Anticancer Drugs 11(7), 555-563 (2000); Huland E, Heinzer H: Renal cell carcinoma—innovative medical treatments. Curr. Opin. Urol. 14(4), 239-244 (2004); Huland E, Burger A, Fleischer J et al.: Efficacy and safety of inhaled recombinant interleukin-2 in high-risk renal cell cancer patients compared with systemic interleukin-2: an outcome study. Folia Biol. (Praha) 49(5), 183-190 (2003); Heinzer H, Mir T, Huland E, Huland H: Subjective and objective prospective, long-term analysis of quality of life during inhaled interleukin-2 immunotherapy. J. Clin. Oncol. 17(11), 3612-3620 (1999); Hartig S, Greene R, Dasgupta J et al.: Multifunctional nanoparticulate polyelectrolyte complexes. Pharm. Res. 24(12), 2353-2369 (2007); Mishra B, Patel B, Tiwari S: Colloidal nanocarriers: a review on formulation technology, types and applications toward targeted drug delivery. Nanomedicine 6(1), 9-24 (2010); Crowder T, Rosati J, Schroeter J, Hickey A, Martonen T: Fundamental effects of particle morphology on lung delivery: predictions of stokes' law and the particular relevance to dry powder inhaler formulation and development. Pharm. Res. 19(3), 239-245 (2002); Oberdorster G, Oberdorster E, Oberdorster J: Nanotoxicology: an emerging discipline evolving from studies of ultrafine particles. Environ. Health Perspect. 113(7), 823-839 (2005); Azarmi S, Tao X, Chen H et al.: Formulation and cytotoxicity of doxorubicin nanoparticles carried by dry powder aerosol particles. Int. J. Pharm. 319(1-2), 155-161 (2006); Vega-Villa K, Takemoto J, Yáñez J, Remsberg C, Forrest M, Davies N: Clinical toxicities of nanocarrier systems. Adv. Drug Deliv. Rev. 60(8), 929-938 (2008); Kawashima Y, Yamamoto H, Takeuchi H, Fujioka S, Hino T: Pulmonary delivery of insulin with nebulized dl-lactide/glycolide copolymer (PLGA) nanospheres to prolong hypoglycemic effect. J. Control. Release 62(1-2), 279-287 (1999); Grenha A, Seijo B, Remuñán-López C: Microencapsulated chitosan nanoparticles for lung protein delivery. Eur. J. Pharm. Sci. 25(4-5), 427-437 (2005); Semmler-Behnke M, Takenaka S, Fertsch S et al.: Efficient elimination of inhaled nanoparticles from the alveolar region: Evidence for interstitial uptake and subsequent reentrainment onto airways epithelium. Environ. Health Perspect. 115(5), 728-733 (2007); Dailey L, Jekel N, Fink L et al.: Investigation of the proinflammatory potential of biodegradable nanoparticle drug delivery systems in the lung. Toxicol. Appl. Pharmacol. 215(1), 100-108 (2006); Azarmi S, Roa W, Lobenberg R: Targeted delivery of nanoparticles for the treatment of lung diseases. Adv. Drug Deliv. Rev. 60(8), 863-875 (2008); Kathuria H, Cao Y, Ramirez M, Williams M: Transcription of the caveolin-1 gene is differentially regulated in lung type I epithelial and endothelial cell lines. A role for ets proteins in epithelial cell expression. J. Biol. Chem. 279(29), 30028-30036 (2004); Roy I, Vij N: Nanodelivery in airway diseases: challenges and therapeutic applications. Nanomedicine 6(2), 237-244 (2010); Brandenberger C, Mühlfeld C, Ali Z et al.: Quantitative evaluation of cellular uptake and trafficking of plain and polyethylene glycol-coated gold nanoparticles. Small 6(15), 1669-1678 (2010); Zhu M, Feng W, Wang Y et al.: Particokinetics and extrapulmonary translocation of intratracheally instilled ferric oxide nanoparticles in rats and the potential health risk assessment. Toxicol. Sci. 107(2), 342-351 (2009); Möller W, Felten K, Sommerer K et al.: Deposition, retention, and translocation of ultrafine particles from the central airways and lung periphery. Am. J. Respir. Crit. Care Med. 177(4), 426-432 (2008); Arppe J, Vidgren M, Waldrep J C: Pulmonary pharmacokinetics of cyclosporin a liposomes. Int. J. Pharm. 161,205-214 (1998); Herber-Jonat S, Mittal R, Gsinn S, Bohnenkamp H, Guenzi E, Schulze A: Comparison of lung accumulation of cationic liposomes in normal rats and lps-treated rats. Inflamm. Res. DOI 10.1007/s00011-010-0260-y (2010) (Epub ahead of print); Videira M, Botelho M, Santos A, Gouveia L, De Lima J, Almeida A: Lymphatic uptake of pulmonary delivered radiolabelled solid lipid nanoparticles. J. Drug Target 10(8), 607-613 (2002); Grenha A, Remuñán-López C, Carvalho E, Seijo B: Microspheres containing lipid/chitosan nanoparticles complexes for pulmonary delivery of therapeutic proteins. Eur. J. Pharm. Biopharm. 69(1), 83-93 (2008); Faraji A, Wipf P: Nanoparticles in cellular drug delivery. Bioorg. Med. Chem. 17(8), 2950-2962 (2009); Huang Y, Wang C: Pulmonary delivery of insulin by liposomal carriers. J. Control. Release 113(1), 9-14 (2006); Bi R, Shao W, Wang Q, Zhang N: Spray-freeze-dried dry powder inhalation of insulin-loaded liposomes for enhanced pulmonary delivery. J. Drug Target 16(9), 639-648 (2008); Chono S, Fukuchi R, Seki T, Morimoto K: Aerosolized liposomes with dipalmitoyl phosphatidylcholine enhance pulmonary insulin delivery. J. Control. Release 137(2), 104-109 (2009); Shahiwala A, Misra A: A preliminary pharmacokinetic study of liposomal leuprolide dry powder inhaler: a technical note. AAPS Pharm Sci Tech 6(3),E482-E486 (2005); Gilbert B, Knight C, Alvarez F et al.: Tolerance of volunteers to cyclosporine a-dilauroylphosphatidylcholine liposome aerosol. Am. J. Respir. Crit. Care Med. 156(6), 1789-1793 (1997); Khanna C, Hasz D, Klausner J, Anderson P: Aerosol delivery of interleukin 2 liposomes is nontoxic and biologically effective: Canine studies. Clin. Cancer Res. 2(4), 721-734 (1996); Khanna C, Anderson P, Hasz D, Katsanis E, Neville M, Klausner J: Interleukin-2 liposome inhalation therapy is safe and effective for dogs with spontaneous pulmonary metastases. Cancer 79(7), 1409-1421 (1997); Ten R, Anderson P, Zein N, Temesgen Z, Clawson M, Weiss W: Interleukin-2 liposomes for primary immune deficiency using the aerosol route. Int. Immunopharmacol. 2(2-3), 333-344 (2002); Kaipel M, Wagner A, Wassermann E et al.: Increased biological half-life of aerosolized liposomal recombinant human Cu/Zn superoxide dismutase in pigs. J. Aerosol Med. Pulm. Drug Deliv. 21(3), 281-290 (2008); Lange C, Hancock R, Samuel J, Finlay W: In vitro aerosol delivery and regional airway surface liquid concentration of a liposomal cationic peptide. J. Pharm. Sci. 90(10), 1647-1657 (2001); Muller R, Petersen R, Hommoss A, Pardeike J: Nanostructured lipid carriers (NLC) in cosmetic dermal products. Adv. Drug Deliv. Rev. 59(6), 522-530 (2007); Liu J, Gong T, Fu H et al.: Solid lipid nanoparticles for pulmonary delivery of insulin. Int. J. Pharm. 356(1-2), 333-344 (2008); Liu Z, Jiao Y, Wang Y, Zhou C, Zhang Z: Polysaccharides-based nanoparticles as drug delivery systems. Adv. Drug Deliv. Rev. 60(15), 1650-1662 (2008); Yang Y, Bajaj N, Xu P, Ohn K, Tsifansky M, Yeo Y: Development of highly porous large plga microparticles for pulmonary drug delivery. Biomaterials 30(10), 1947-1953 (2009); Huang X, Du Y, Yuan H, Hu F: Preparation and pharmacodynamics of low-molecular-weight chitosan nanoparticles containing insulin. Carbohydr. Poly. 76,368-373 (2009); Yamamoto H, Hoshina W, Kurashima H et al.: Engineering of poly(dl-lactic-co-glycolic acid) nanocomposite particles for dry powder inhalation dosage forms of insulin with the spray-fluidized bed granulating system. Adv. Powder Technol. 18(2), 215-228 (2007); Grenha A: Microencapsulación de nanopartículas de quitosano para la administración pulmonar de macromoléculas terapéuticas. Universidad de Santiago de Compostela, Facultad de Farmacia, Departamento de Farmacia y Tecnologia Farmacéutica. PhD Thesis, 306 (2006); Zhang Q, Shen Z, Nagai T: Prolonged hypoglycemic effect of insulin-loaded polybutylcyanoacrylate nanoparticles after pulmonary administration to normal rats. Int. J. Pharm. 218(1-2), 75-80 (2001); Kawashima Y, Yamamoto H, Takeuchi H, Kuno Y: Mucoadhesive DL/glycolide copolymer nanospheres coated with chitosan to improve oral delivery of elcatonin. Pharm. Dev. Technol. 5(1), 77-85 (2000); Yamamoto H, Kuno Y, Sugimoto S, Takeuchi H, Kawashima Y: Surface-modified plga nanosphere with chitosan improved pulmonary delivery of calcitonin by mucoadhesion and opening of the intercellular tight junctions. J. Control. Release 102(2), 373-381 (2005); Renwick L, Brown D, Clouter A, Donaldson K: Increased inflammation and altered macrophage chemotactic responses caused by two ultrafine particle types. Occup. Environ. Med. 61(5), 442-447 (2004); Chang C: The immune effects of naturally occurring and synthetic nanoparticles. J. Autoimmun. 34(3),J234-J246 (2010); Thomas D, Myers M, Wichert B, Schreier H, Gonzalez-Rothi R: Acute effects of liposome aerosol inhalation on pulmonary function in healthy human volunteers. Chest 99(5), 1268-1270 (1991); Lizio R, Klenner T, Sarlikiotis A et al.: Systemic delivery of cetrorelix to rats by a new aerosol delivery system. Pharm. Res. 18(6), 771-779 (2001); Niven R, Whitcomb K, Woodward M, Liu J, Jornacion C: Systemic absorption and activity of recombinant consensus interferons after intratracheal instillation and aerosol administration. Pharm. Res. 12(12), 1889-1895 (1995); Taljanski W, Pierzynowski S, Lundin P et al.: Pulmonary delivery of intratracheally instilled and aerosolized cyclosporine a to young and adult rats. Drug Metab. Dispos. 25(8), 917-920 (1997); Bennett D, Tyson E, Nerenberg C, Mah S, De Groot J, Teitelbaum Z: Pulmonary delivery of detirelix by intratracheal instillation and aerosol inhalation in the briefly anesthetized dog. Pharm. Res. 11(7), 1048-1055 (1994); Schreier H, Mcnicol K, Bennett D, Teitelbaum Z, Derendorf H: Pharmacokinetics of detirelix following intratracheal instillation and aerosol inhalation in the unanesthetized awake sheep. Pharm. Res. 11(7), 1056-1059 (1994); Patton J S, Trinchero P, Platz R M: Bioavailability of pulmonary delivered peptides and proteins—α-interferon, calcitonins and parathyroid hormones. J. Control. Release 28(1-3), 79-85 (1994); Colthorpe P, Farr S, Smith I, Wyatt D, Taylor G: The influence of regional deposition on the pharmacokinetics of pulmonary-delivered human growth hormone in rabbits. Pharm. Res. 12(3), 356-359 (1995); Pfutzner A, Mann A, Steiner S: Technosphere/insulin—a new approach for effective delivery of human insulin via the pulmonary route. Diabetes Technol. Ther. 4(5), 589-594 (2002); Pang Y, Sakagami M, Byron P: The pharmacokinetics of pulmonary insulin in the in vitro isolated perfused rat lung: implications of metabolism and regional deposition. Eur. J. Pharm. Sci. 25(4-5), 369-378 (2005); Rave K, Bott S, Heinemann L et al.: Time-action profile of inhaled insulin in comparison with subcutaneously injected insulin lispro and regular human insulin. Diabetes Care 28(5), 1077-1082 (2005); Niven R, Lott F, Cribbs J: Pulmonary absorption of recombinant methionyl human granulocyte colony stimulating factor (r-hug-csf) after intratracheal instillation to the hamster. Pharm. Res. 10(11), 1604-1610 (1993); Hubbard R, Crystal R: Strategies for aerosol therapy of α1-antitrypsin deficiency by the aerosol route. Lung 168(Suppl.), 565-578 (1990); Kobayashi S, Kondo S, Juni K: Pulmonary delivery of salmon calcitonin dry powders containing absorption enhancers in rats. Pharm. Res. 13(1), 80-83 (1996); Lizio R, Klenner T, Borchard G et al.: Systemic delivery of the gnrh antagonist cetrorelix by intratracheal instillation in anesthetized rats. Eur. J. Pharm. Sci. 9(3), 253-258 (2000); Iacono A, Johnson B, Grgurich W et al.: A randomized trial of inhaled cyclosporine in lung-transplant recipients. N. Engl. J. Med. 354(2), 141-150 (2006); Folkesson H, Weström B, Dahlbäck M, Lundin S, Karlsson B: Passage of aerosolized bsa and the nona-peptide dDAVP via the respiratory tract in young and adult rats. Exp. Lung Res. 18(5), 595-614 (1992); Christopher F, Chase D, Stein K, Milne R: Rhdnase therapy for the treatment of cystic fibrosis patients with mild to moderate lung disease. J. Clin. Pharm. Ther. 24(6), 415-426 (1999); Bitonti A, Dumont J, Low S et al.: Pulmonary delivery of an erythropoietin fc fusion protein in non-human primates through an immunoglobulin transport pathway. Proc. Natl Acad. Sci. USA 101(26), 9763-9768 (2004); Russell K, Read M, Bellinger D et al.: Intratracheal administration of recombinant human factor ix (benefix) achieves therapeutic levels in hemophilia b dogs. Thromb. Haemost. 85(3), 445-449 (2001); Onoue S, Yamamoto K, Kawabata Y, Hirose M, Mizumoto T, Yamada S: Novel dry powder inhaler formulation of glucagon with addition of citric acid for enhanced pulmonary delivery. Int. J. Pharm. 382(1-2), 144-150 (2009); Low S, Nunes S, Bitonti A, Dumont J: Oral and pulmonary delivery of fsh-fc fusion proteins via neonatal fc receptor-mediated transcytosis. Hum. Reprod. 20(7), 1805-1813 (2005); Codrons V, Vanderbist F, Ucakar B, Préat V, Vanbever R: Impact of formulation and methods of pulmonary delivery on absorption of parathyroid hormone (1-34) from rat lungs. J. Pharm. Sci. 93(5), 1241-1252 (2004); Codrons V, Vanderbist F, Verbeeck R et al.: Systemic delivery of parathyroid hormone (1-34) using inhalation dry powders in rats. J. Pharm. Sci. 92(5), 938-950 (2003); Van Zandwijk N, Jassem E, Dubbelmann R, Braat M, Rumke P: Aerosol application of interferon-α in the treatment of bronchioloalveolar carcinoma. Eur. J. Cancer 26(6), 738-740 (1990); Martin P, Vaidyanathan S, Lane J et al.: Safety and systemic absorption of pulmonary delivered human ifn-β1a in the nonhuman primate: comparison with subcutaneous dosing. J. Interferon Cytokine Res. 22(6), 709-717 (2002); Halme M, Maasilta P, Repo H et al.: Inhaled recombinant interferon-γ in patients with lung cancer: pharmacokinetics and effects on chemiluminescence responses of alveolar macrophages and peripheral blood neutrophils and monocytes. Int. J. Radiat. Oncol. Biol. Phys. 31(1), 93-101 (1995); Eichelberg C, Andreas A, Heuer R, Huland H, Heinzer H, Huland E: Long-term tumor control with inhalational interleukin 2 therapy in cardiac high risk patients with metastatic renal carcinoma. J. Urol. 179(4), 167-167 (2008); Adjei A, Sundberg D, Miller J, Chun A: Bioavailability of leuprolide acetate following nasal and inhalation delivery to rats and healthy humans. Pharm. Res. 9(2), 244-249 (1992); Adjei A, Garren J: Pulmonary delivery of peptide drugs: effect of particle size on bioavailability of leuprolide acetate in healthy male volunteers. Pharm. Res. 7(6), 565-569 (1990); Tazawa R, Nakata K, Inoue Y, Nukiwa T: Granulocyte-macrophage colony-stimulating factor inhalation therapy for patients with idiopathic pulmonary alveolar proteinosis: a pilot study; and long-term treatment with aerosolized granulocyte-macrophage colony-stimulating factor: a case report. Respirology11(Suppl.) S61-S64 (2006); US2006/0120969 (2006), each of which is expressly incorporated herein by reference.

One aspect of the cancer stem cell hypothesis is that patients with tumors that exhibit stem-like phenotypes have poor prognoses. Distal epithelial progenitors from lungs early in development demonstrate both self-renewal and potential to differentiate into all bronchial and alveolar epithelial cell types. By contrast, late progenitors are only able to produce alveolar cells. The various differentiated cell types have different gene expression characteristics. Low L-PGDS expression has been found associated (along with 9 other genes) with a predicted poor prognosis in human lung cancer. “A 10-Gene Progenitor Cell Signature Predicts Poor Prognosis in Lung Adenocarcinoma” Onaitis M, D'Amico T A, Clark C P, Guinney J, Harpole D H, Rawlins E L, Ann Thorac Surg 2011; 91:1046-50.

### EXAMPLE 1

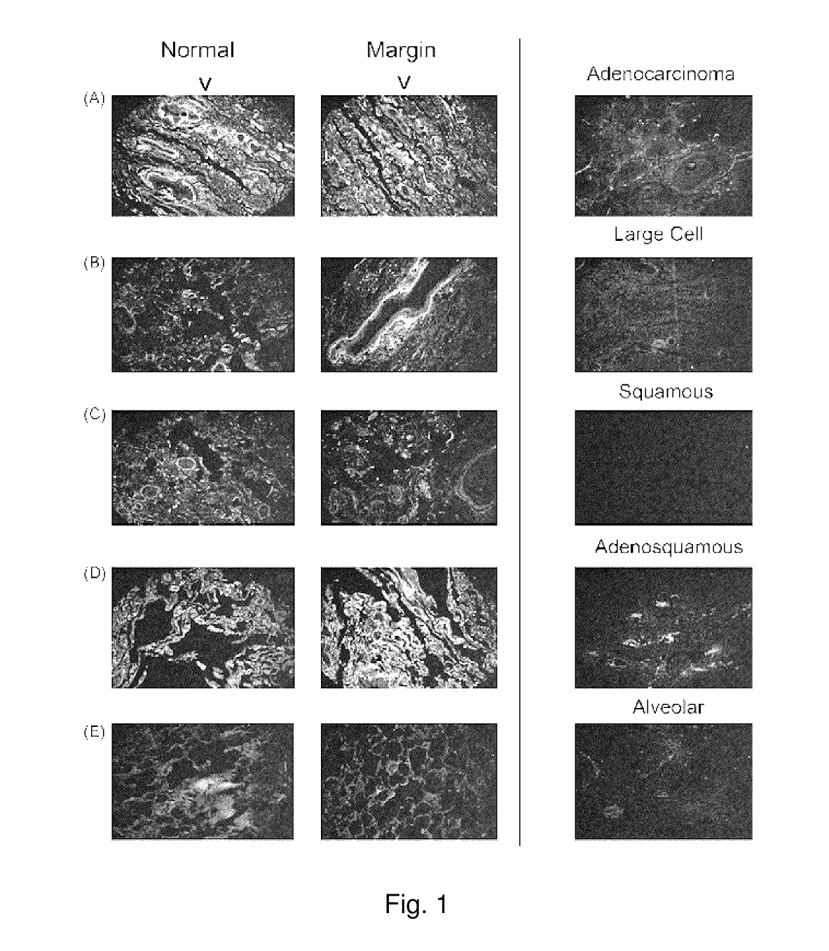

Given available L-PGDS data involving apoptosis, cell cycle and migration in various cell types, as well as the preponderance of data linking L-PGDS to cancer, the expression of L-PGDS in tumor versus normal tissue as well as the margin of various types of lung cancer was investigated. As can be seen in FIG. 1A-1E, immunohistochemical fluorescence staining shows significantly less L-PGDS protein expression in human non-small cell lung cancer (NSCLC) tumor types as compared to normal margins.

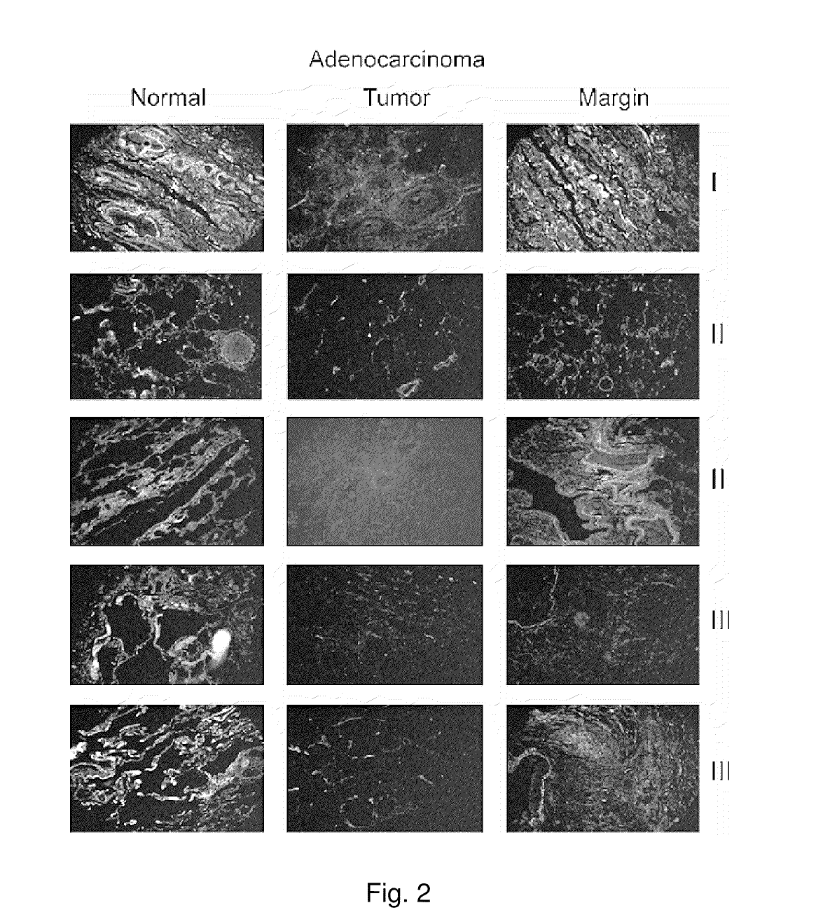

Furthermore, lung tissue sections with varying grades adenocarcinoma revealed that the fluorescence L-PGDS staining in the tumor tissue was significantly lower than margin and decreased with grade progression, as shown in FIG. 2.

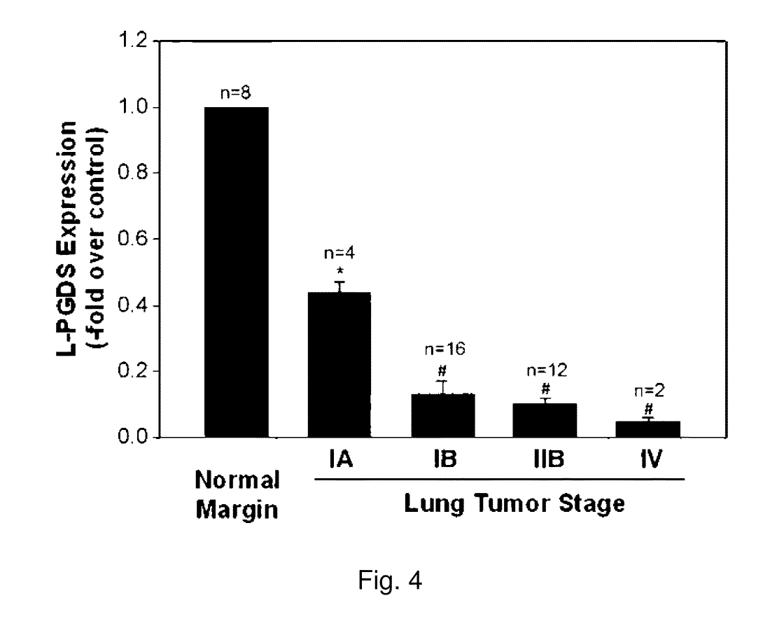

L-PGDS gene expression in human lung tumors of various grades (Ia, Ib, IIb, and IV) was examined by RT-PCR using a Tissue Scan Lung Cancer Tissue qPCR Array I (Origene Technologies, Rockville, Md., Part #HLRT101). L-PGDS gene expression decreased proportionately with the stage of tumor progression when compared to normal lung tissue, as shown in FIG. 4.

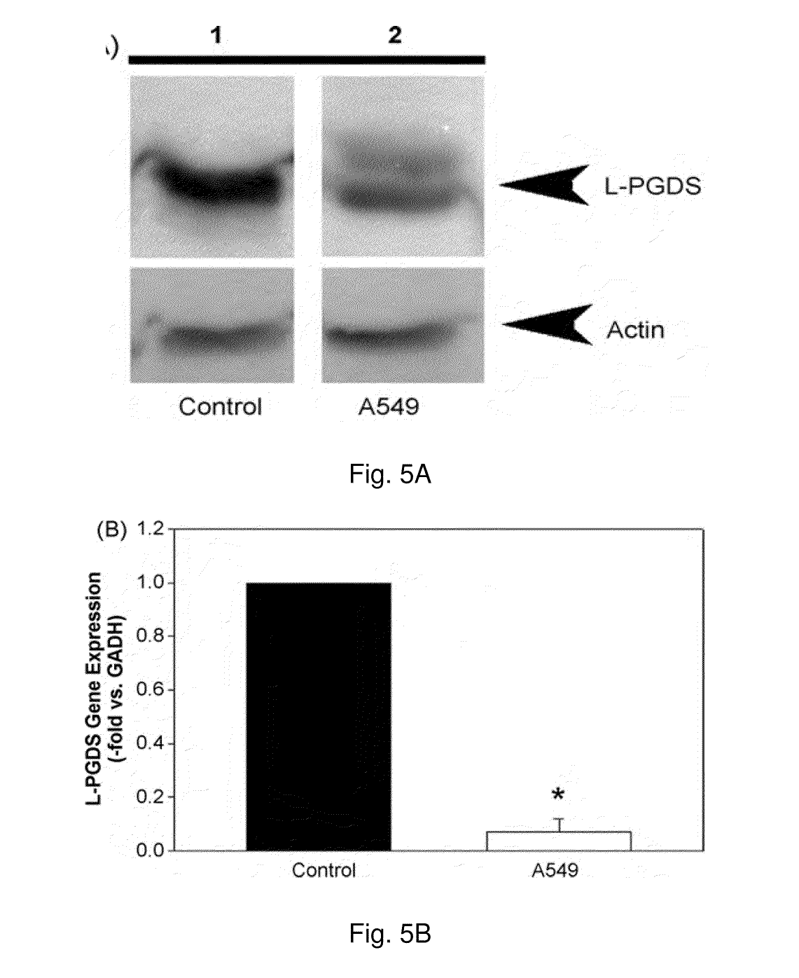

Experiments were performed in culture of both control mouse lung epithelial (MLE) cells and the carcinomic human alveolar basal epithelial cell line (A549) to assess both L-PGDS gene and protein expression. As can be seen in the right side of FIG. 5B, there is a 10-fold decrease in L-PGDS gene expression in A549 when compared to MLE as determined by RT-PCR. In addition, a 6-fold decrease in L-PGDS protein expression was observed in A549 when compared to controls.

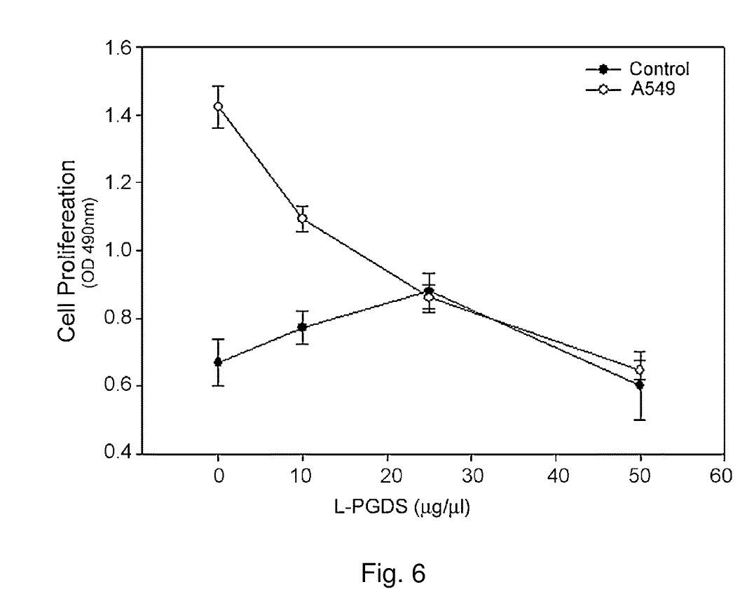

The effect of exogenously added L-PGDS on lung cell growth in culture was determined by MTS proliferation assay (Promega, Madison, Wis.). A marked inhibition of A549 cell proliferation over the entire range of L-PGDS (1-50 mg/l) was observed after 18 h incubation. MLE cell proliferation was unaffected at L-PGDS concentrations up to 10 mg/dl but ceased at a concentration of 25 mg/l and above, as shown in FIG. 6.

Elevated L-PGDS expression has been shown to help minimize tumor growth and L-PGDS knockout mice will be more susceptible to tumor growth than normal mice.

Cell culture reagents, including fetal bovine serum and platelet derived growth factor-BB (PDGF), were all purchased from Life Technologies (Grand Island, N.Y.). SDS/polyacrylamide gel electrophoresis and western blot reagents were from Bio-Rad (Hercules, Calif.). Bicinchoninic acid protein assay reagent was purchased from Pierce (Rockford, Ill.). Western blots were visualized with enhanced chemiluminescence reagent purchased from Amersham Pharmacia Biotech (Piscataway, N.J.). Type-1 collagenase was from Worthington Biochemical Co. (Freehold, N.J.). All other reagents were purchased from the Sigma Chemical Co. (St. Louis, Mo.).

The Normal Lung Tissue Array (NC04-01) and Lung Adenocarcinoma Tissue Array (C504-08) were purchased from CYBRDI Inc. (Rockville, Md.). TissueScan Real Time Cancer Expression Panel (HLRT 101) was purchased from Origene (Rockville, Md.). In addition, we procured tumor and surrounding margin from eight lung adenocarcinomas to help support the commercial tissue array data.

Sections were deparafinized in a xylene/ethanol series; antigen unmasked in 10 mM citric acid and blocked in 10% goat serum in PBS. The primary antibody was an L-PGDS monoclonal raised in rat (Cayman, Ann Arbor, Mich.) diluted 1:1000 in 1.5% goat serum and incubated overnight at 4° C. The secondary antibody was a FITC labeled goat anti-rat (Santa Cruz Biotechnology, Santa Cruz, Calif.) diluted 1:20 in 10% goat serum. Sections were visualized with a Nikon Eclipse TE 300 fluorescent microscope. Control slides were incubated with nonspecific primary anti-sera or in some cases without the primary antibody. In no case did controls show a positive signal.

Typically, 50 μg of whole cell protein lysate was mixed with Laemmli sample buffer containing 0.1% bromophenol blue; 1.0M NaH2PO4, pH 7.0; 50% glycerol and 10% SDS, boiled for 5 min and loaded on an SDS 10% polyacrylamide gel. The separated proteins were transferred to polyvinylidene difluoride membrane (Bio-Rad, Hercules, Calif.), probed with the proper primary antibodies followed by 1:2000 diluted secondary antibody and detected with enhanced chemiluminescence reagent (GE Healthcare, Piscataway, N.J.) and subsequent autoradiography. The intensity of the signal was quantitated by densitometric analysis using SigmaGel 1.0 software (Jandel/Systat Inc., San Jose, Calif.).

RNA was isolated from confluent cells or lung tissue with Trizol (Invitrogen, Carlsbad, Calif.) according to the manufacturer's protocol. First strand cDNAs were prepared from 2 μg RNA of each sample using the Transcriptor High Fidelity cDNA Synthesis Kit for RT-PCR (Roche, Indianapolis, Ind.) according to the manufacturer's protocol. Primers for targets L-PGDS or the reference GAPDH were designed with Beacon Designer software using sequences found in the NCBI gene bank based on the criteria that they were at least 20 nucleotides in length had a Tm of approximately 60° C., and an amplicon length of between 75 and 200 bp.

| L-PGDS: | |
|---|---|
| SEQ ID NO: 001 | |
| Forward | 5′GAA GAA GGC GGC GTT GTC-3′, |
| SEQ ID NO: 002 | |
| Reverse | 5′-GAG GAA GGT GGA GGT CAG G-3′; |
| GAPDH: | |
| SEQ ID NO: 003 | |
| Forward | 5′-GCT CTC TGC TCC TCC TGT TC-3′, |
| SEQ ID NO: 004 | |
| Reverse | 5′-GAC TCC GAC CTT CAC CTT CC-3′. |

Quantitative RT-PCR was performed in duplicate on a LightCycler 480 using the Syber-Green I Master Mix (Roche). A final reaction volume of 20 μl containing 2 μl cDNA, 10 μl MasterMix, 1 μl of each primer (0.5 μM), and 6 μl of PCR grade water was used. The reaction was performed with a denaturation step of 95° C. for 5 min followed by 40 cycles at 95° C. for 45 s, 61° C. for 1 min, and 72° C. for 1 min. For the creation of standard curves, cDNA from a test sample was diluted 1:3, followed by a 5-fold dilution to yield a final 1:375 dilution. All reactions were run with a negative control and subjected to melting curve analysis. Fold changes in gene expression were calculated using the Pfaffl method.

A549, a human non-small cell carcinoma line that exhibits type II-like alveolar epithelial characteristics was obtained from the American Type Culture Collection, and cultured in RPMI and supplemented with 5% fetal bovine serum (FBS), 100 U/ml penicillin and 100 μg/ml streptomycin, and grown in a humidified atmosphere of 5% CO2 at 37° C.

### Cell Proliferation Assay

Cell proliferation was measured with the use of a CellTiter 96 AQeous Non-Radioactive Cell Proliferation Assay (Promega, Madison, Wis.). Briefly, cells were plated in 96-well plates at a density of 500 cells/well in 200 μl of medium with 5% FBS. The cells were incubated at 37° C. in a humidified 5% CO2 atmosphere for the indicated time, after which 20 μl of combined MTS/PMS solution (Promega) was added per well. After a 2 h incubation at 37° C., absorbance at 490 nm was measured using an enzyme-linked immunosorbent assay (ELISA) plate reader. Data represent the average absorbance of three experiments each performed in triplicate.

Migration assays were performed using 24-well cell culture inserts with 8.0 μm polyethylene terephthalate cyclopore membranes (Falcon) as detailed in Lundberg M S, Curto K A, Bilato C, Monticone R E, Crow M T. Regulation of vascular smooth muscle migration by mitogen-activated protein kinase and calcium/calmodulin-dependent protein kinase II signaling pathways. J Mol Cell Cardiol 1998; 30:2377-89. The underside of the membrane was coated with 100 μl rat tail collagen type I (50 μg/ml) for 18-20 h, washed and air-dried before each experiment. Cells were trypsinized and re-suspended in RPMI. Then 2×104 cells/250 μl were loaded into the cell culture inserts. The inserts were then added to the wells of 24-well plates which were filled with PDGF-BB diluted in RPMI with 0.1% BSA, and where indicated, L-PGDS was also added to this medium below the inserts.

The chambers were then incubated at 37° C. for 5 h to allow for cell migration. Afterwards, cells were completely removed from the upper side of the membrane with a cotton swab and the remaining migrated cells fixed and stained with Diff-Quik® solution (Dade Behring, Newark, Del.). Results are reported as the mean+/−SEM of five different fields, from three experiments, counted at 200× magnification.

Experimental results are reported as means±SEM. Data was analyzed by one-way ANOVA for comparisons of multiple data sets and by Student's t-test for comparison of two data sets using SigmaStat 3.5 (Systat), with statistical significance set at P<0.05.

[000102] Given previous L-PGDS data involving apoptosis, cell cycle and migration in various cell types, as well as the preponderance of data linking L-PGDS to cancer; the expression of L-PGDS was investigated in lung tumor versus normal tissue as well as the margin of various types of lung cancer using a commercially available tissue array.

FIGS. 1A-1E presents Immunohistochemical fluorescence staining images, which show significantly less L-PGDS protein expression in human non-small cell lung cancer (NSCLC) tumor types when compared to normal lung tissue and tumor margins. The Normal Lung Tissue Array (NC04-01) purchased from CYBRDI Inc. (Rockville, Md.) was treated as described above and incubated with monoclonal L-PGDS antibody diluted 1:1000 followed by FITC-labeled goat anti-rat (Santa Cruz Biotechnology, Santa Cruz, Calif.) diluted 1:20 in 10% goat serum. Sections were visualized with a Nikon Eclipse TE 300 fluorescent microscope at 200× magnification.

Differences between normal/margin and tumor L-PGDS expression were most pronounced in adenocarcinoma and adenosquamous cells (FIGS. 1A and D, respectively).

Analysis of a lung tissue array of various stages adenocarcioma demonstrated a decrease in L-PGDS expression with increasing tumor stage, as shown in FIG. 2. The fluorescence stained images of FIG. 2 show a Lung Adenocarcinoma Tissue Array (C504-08) purchased from CYBRDI Inc. (Rockville, Md.) which was treated as described above and incubated with monoclonal L-PGDS antibody diluted 1:1000 followed by FITC-labeled goat anti-rat (Santa Cruz Biotechnology, Santa Cruz, Calif.) diluted 1:20 in 10% goat serum. Sections were visualized with a Nikon Eclipse TE 300 fluorescent microscope at 200× magnification.

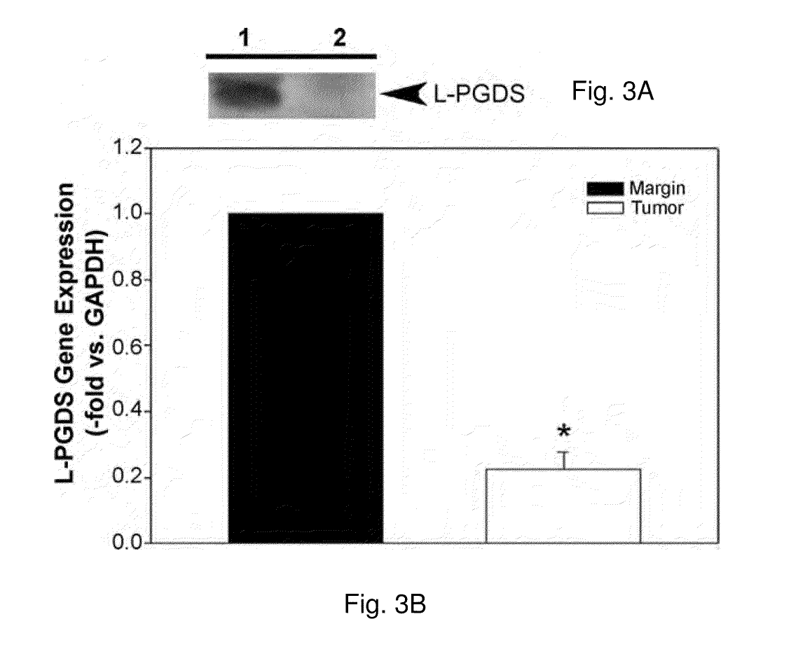

The absence of L-PGDS protein expression in ten lung adenocarcinoma and corresponding margin homogenates. A representative western blot is provided in FIG. 3A. There was an unambiguous absence of L-PGDS protein expression in the tumor when compared to healthy margin along with an average 8-fold decrease in L-PGDS gene expression, as shown in FIG. 3B. Protein lysates (50 μg) were isolated from an adenocarcinoma (lane 1) and corresponding margin (lane 2) and subjected to Western analysis to quantify L-PGDS protein expression as described above (FIG. 3A). First strand cDNAs were prepared from 2.5 μg RNA of each sample and L-PGDS gene expression, expressed as -fold versus GAPDH, was examined by quantitative RT-PCR performed in triplicate on a LightCycler 480 as described above, see FIG. 3B. Asterisk (*) represents P<0.01 versus control.

L-PGDS gene expression was examined in a variety of human lung tumors of various grades (Ia, Ib, IIb, and IV) by RTPCR using a TissueScan Lung Cancer Tissue qPCR Array I (Origene Technologies, Rockville, Md., Part #HLRT101). LPGDS gene expression decreased proportionately with the stage of tumor progression when compared to normal lung tissue (FIG. 4).

FIG. 4 shows L-PGDS gene expression decreases with tumor progression. A TissueScan Real Time Cancer Expression Panel (HLRT 101) from Origene Tech., Inc. (Rockville, Md.) was used to quantify L-PGDS expression. Sections were processed according to manufacturer's instructions and quantitative RT-PCR performed in duplicate on a Roche LightCycler 480 using a Sybr-Green I Master Mix (Roche). The reaction was performed with a denaturation step of 50° C. for 2 min then 95° C. for 5 min followed by 40 cycles at 95° C. for 45 s, 61° C. for 1 min, and 72° C. for 1 min with primers described in Section 2. All reactions were run with a negative control and subjected to melting curve analysis. Fold changes in gene expression were calculated using the Pfaffl method.

Similar patterns of L-PGDS protein and gene expressions were observed in cultured lung epithelial cells. As seen in FIG. 5A, there was a 4-fold decrease in L-PGDS protein expression in the human non-small cell carcinoma A549 cell line when compared to controls.

Similarly, there was also a 10-fold decrease in L-PGDS gene expression in the A549 cells compared to controls as determined by RT-PCR (FIG. 5B).

FIG. 5B thus shows L-PGDS expression in A549 cells. Protein lysates (50 μg) were isolated from cultured cells and subjected to Western analysis to quantify L-PGDS protein expression as described above (panel A). The expression of μ-actin was used to normalize L-PGDS expression. First strand cDNAs were prepared from 2.5 μg RNA of each sample and L-PGDS gene expression, expressed as -fold versus GAPDH, was monitored via quantitative RT-PCR performed in duplicate on a LightCycler 480 as described above (panel B). Asterisk (*) represents P<0.01 versus control.

The effect of exogenously added L-PGDS on A549 proliferation was observed. There was a dose responsive inhibition of A549 hyper-proliferative growth (proliferation determined after 2 h) in the presence of exogenously added L-PGDS (0, 10, 25, 50 μg/ml) which mimicked control cells after a concentration of 25 μg/μl expressed as OD490nm, as shown in FIG. 6.

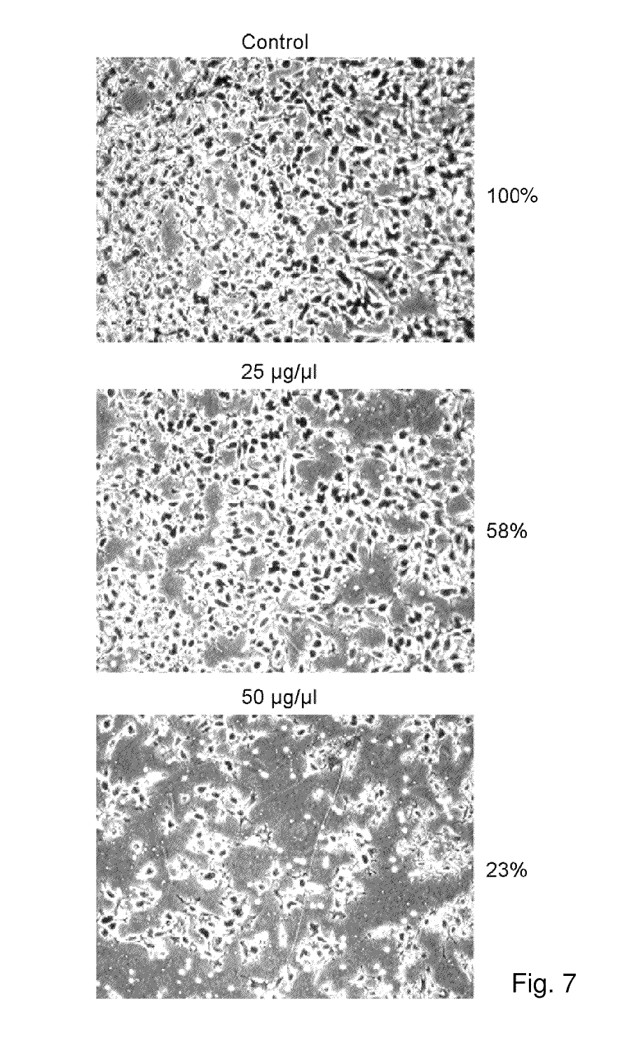

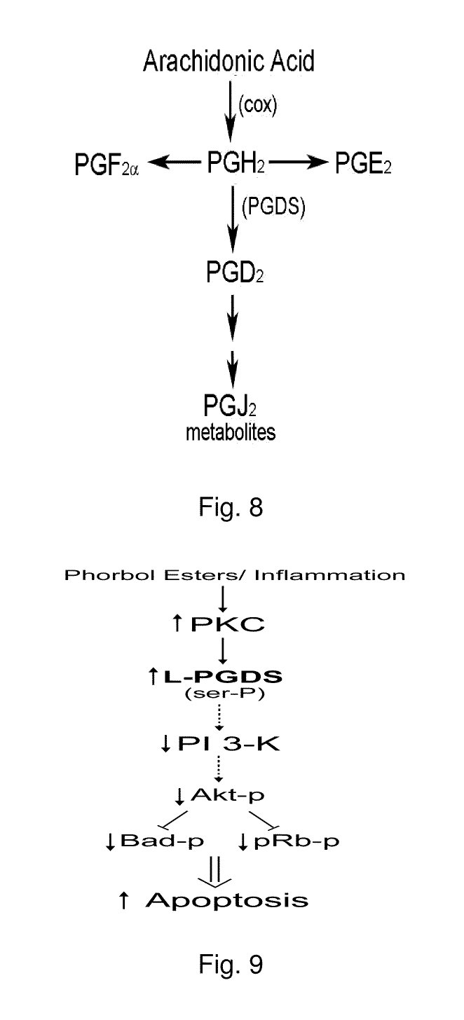

The effect of L-PGDS on A549 migration was examined using trans-well inserts. FIG. 7 illustrates a 42% decrease in PGDF stimulated A549 migration in the presence of 25 μg/μl L-PGDS and a 77% decrease in the presence of 50 μg/μl L-PGDS. The addition of 100 μg/μl L-PGDS offered no further increase in migration inhibition (data not shown) and would support the notion that the L-PGDS effect is primarily due to alteration in migration as opposed to apoptotic influences. Cells were cultured and loaded into collagen coated 12-well cell culture inserts in the absence or presence of L-PGDS (25 or 50 μg/ml) as described above. Actual fields at 200× magnification are presented.

L-PGDS induces cellular apoptosis, delaying cell cycle progression, and inhibiting cell proliferation and migration in multiple cell types. Ragolia L, Palaia T, Paric E, Maesaka J K. Prostaglandin D2 synthase inhibits the exaggerated growth phenotype of spontaneously hypertensive rat vascular smooth muscle cells. J Biol Chem 2003; 278:22175-81; Ragolia L, Palaia T, Koutrouby T B, Maesaka J K. Inhibition of cell cycle progression and migration of vascular smooth muscle cells by prostaglandin D2 synthase: resistance in diabetic Goto-Kakizaki rats. Am J Physiol Cell Physiol 2004; 287:C1273-81; Maesaka J K, Palaia T, Frese L, Fishbane S, Ragolia L. Prostaglandin D(2) synthase induces apoptosis in pig kidney LLC-PK1 cells. Kidney Int 2001; 60:1692-8. The absence of L-PGDS protein and gene expression in NSCLC was demonstrated (FIGS. 1-3). Furthermore, an inverse relationship between L-PGDS gene expression and lung tumor progression was observed (FIG. 4). The use of lung tissue arrays allows a comparison of a wide range of lung tumor samples which was consistent with the data obtained from pathological tissue samples.

A549 cells were used as a model cell line to examine basal L-PGDS expression as well as the effect of exogenously added L-PGDS on cell proliferation and migration. Interestingly, basal L-PGDS expression was lower in A549 cells when compared to controls (FIG. 5). Exogenously added LPGDS was able to suppress A549 hyperproliferation and migration (FIGS. 6 and 7), supporting a mechanistic role in lung tumor progression for L-PGDS.

The development of more effective chemopreventive and chemotherapeutic agents with minimal toxicity to treat lung cancer is crucial. Recent efforts have attempted to illustrate the importance of identifying the molecular mechanisms by which PGs promotes tumor growth and metastasis in order to develop safer strategies for cancer prevention and treatment. Wang D, Dubois R N. Prostaglandins and cancer. Gut 2006; 55:115-22. The initial excitement of using COX-2 inhibitors as practical chemopreventives was dampened by the undesirable cardiovascular side effects observed after prolonged use Rahme E, Nedjar H. Risks and benefits of COX-2 inhibitors vs non-selective NSAIDs: does their cardiovascular risk exceed their gastrointestinal benefit? A retrospective cohort study. Rheumatology (Oxford) 2007; 46:435-8; Solomon S D, McMurray J J, Pfeffer M A, Wittes J, Fowler R, Finn P, et al. Cardiovascular risk associated with celecoxib in a clinical trial for colorectal adenoma prevention. N Engl J Med 2005; 352:1071-80. While it appears that such broad inhibition of PG synthesis may be too far upstream, regulation of L-PGDS, a point downstream of PGH2, may provide the mechanism necessary for fine-tuning PG signaling in a temporal and tissue-specific manner and offer a more efficacious chemotherapeutic site for the prevention/treatment of lung cancer. The balance between PGE synthase and L-PGDS, which incidentally has been described as a major determinant of other disease processes such as atherosclerosis, Cipollone F, Fazia M, Iezzi A, Ciabattoni G, Pini B, Cuccurullo C, et al. Balance between PGD synthase and PGE synthase is a major determinant of atherosclerotic plaque instability in humans. Arterioscler Thromb Vasc Biol 2004; 24:1259-65, also plays a significant role in lung cancer progression.

Present studies indicate possible imbalanced expression of prostanoid receptors in colorectal cancer compared to normal colon tissue. Gustafsson A, Hansson E, Kressner U, Nordgren S, Andersson M, Lonnroth C, et al. Prostanoid receptor expression in colorectal cancer related to tumor stage, differentiation and progression. Acta Oncol 2007; 46:1107-12. L-PGDS or synthetic DP1 receptor agonists for its enzymatic product prostaglandin D₂ represent very attractive downstream sites for the prevention or treatment of lung cancer. L-PGDS may thus play a key role in modulating lung cancer growth and may offer a novel diagnostic and therapeutic approach for treatment.

### EXAMPLE 2

L-PGDS knockout mice were originally obtained from the Osaka Bioscience Institute (Osaka, Japan). Eguchi N, Minami T, Shirafuji N, Kanaoka Y, Tanaka T, Nagata A, Yoshida N, Urade Y, Ito S, Hayaishi O, “Lack of tactile pain (allodynia) in lipocalin-type prostaglandin D synthase-deficient mice”, Proc Natl Acad Sci USA 96:726-730 (1999).

Transgenic L-PGDS overexpressors, were purchased from Jackson Laboratories (Bar Harbor, Me.). Hayaishi O, “Molecular genetic studies on sleep-wake regulation, with special emphasis on the prostaglandin D(2) system”, J Appl Physiol 92:863-868 (2002).

Control C57BL/6 mice were purchased from Jackson Laboratories (Bar Harbor, Me.).

Mice are maintained in temperature-controlled rooms (22° C.) with a 12 h light/dark cycle and given access to Rodent Lab Chow, #5001 (Purina, St. Louis, Mo.) and water ad libitum. Experiments are performed on 7-9 week old males housed 4 per cage, in plastic cages with hardwood bedding and dust covers following a 7-day quarantine.

The efficacy of DP1/DP2 receptor agonists were tested, alone and in combination, on lung tumor formation in control and LPGDS KO mice in two separate experiments. The results of this investigation were inconclusive. While DP1 agonist may offer slight protection, the result was not reproducible within the experimental model employed. In addition the incubation conditions may need to be changed, for example, from 1 hr prior to tumor induction to several doses over the two week period. See, Wang J J, Mak O T, “Induction of apoptosis in non-small cell lung carcinoma A549 cells by PGD2 metabolite, 15d-PGD2”, Cell Biol Int. 2011 Apr. 21.

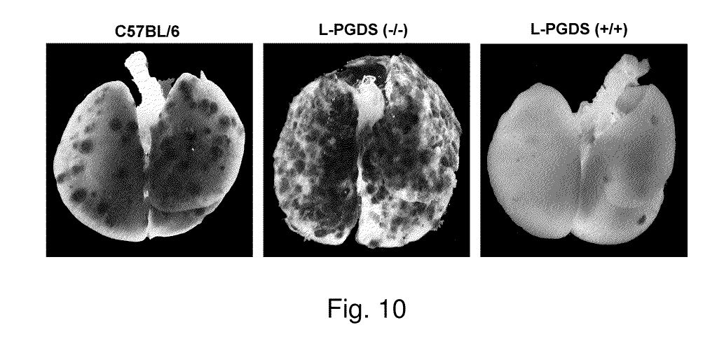

In another experiment, shown in FIG. 10, a mouse melanoma cell line was injected into three different strains of mice—Control C57BL/6, L-PGDS Knockouts, transgenic L-PGDS overexpressors. After 14 days the lungs were isolated and photographed. The L-PGDS overexpressors had low tumor count, while the L-PGDS Knockouts had a large visible number of tumors. The control C57BL/6 mice were intermediate.

### EXAMPLE 3

Another aspect of the invention provides a method for intra-tracheal gene therapy for lung cancer treatment. According to this method, a gene vector is produced using adenovirus or SV-40 viral gene delivery technology, for example of the L-PGDS gene, via intra-tracheal delivery, for example. See, e.g, U.S. Pat. Nos. 7,951,785, 7,960,525, 7,943,374, expressly incorporated herein by reference. See also: Acsadi G et al. “Human dystrophin expression in mdx mice after intramuscular injection of DNA constructs.” Nature 352:815-18 (1991); Armentano D et al. “Expression of human factor IX in rabbit hepatocytes by retrovirus-mediated gene transfer: Potential for gene therapy of hemophilia B.” Proc. Natl. Acad. Sci. USA 87:6141-45 (1990); Berkner K L. “Development of Adenovirus Vectors for the Expression of Heterologous Genes.” Biotechniques 6(7):616-29 (1988); Better M et al. “Escherichia coli Secretion of an Active Chimeric Antibody Fragment.” Science 240:1041-43 (1988); Chowdhury J R et al., “Long-Term Improvement of Hypercholesterolemia After ex Vivo Gene Therapy in LDLR-Deficient Rabbits.” Science 254:1802-05 (1991); Cole et al. (1985), “The EBV-Hybridoma Technique and its Application to Human Lung Cancer.” Monoclonal Antibodies and Cancer Therapy, Alan R. Liss, Inc., pp. 77-96; Danos O and Mulligan R C, “Safe and efficient generation of recombinant retroviruses with amphotropic and ecotropic host ranges.” Proc. Natl. Acad. Sci. USA 85:6460-64 (1988); Eglitis M A et al., “Gene Expression in Mice After High Efficiency Retroviral-Mediated Gene Transfer.” Science 230:1395-98 (1985); Ferry N et al., “Retroviral-mediated gene transfer into hepatocytes in vivo.” Proc. Natl. Acad. Sci. USA 88:8377-81 (1991); Flotte T R et al., “Gene Expression from Adeno-associated Virus Vectors in Airway Epithelial Cells.” Am. J. Respir. Cell. Mol. Biol. 7:349-56 (1992); Flotte T R et al., “Expression of the Cystic Fibrosis Transmembrane Conductance Regulator from a Novel Adeno-associated Virus Promoter.” J. Biol. Chem. 268(5):3781-90 (1993); Haj-Ahmad Y and Graham F L, “Development of a Helper-Independent Human Adenovirus Vector and Its Use in the Transfer of the Herpes Simplex Virus Thymidine Kinase Gene.” J. Virol. 57(1):267-74 (1986); Hermonat P L and Muzyczka N, “Use of adeno-associated virus as a mammalian DNA cloning vector: Transduction of neomycin resistance into mammalian tissue culture cells.” Proc. Natl. Acad. Sci. USA 81:6466-70 (1984); Herz J and Gerard R D, “Adenovirus-mediated transfer of low density lipoprotein receptor gene acutely accelerates cholesterol clearance in normal mice.” Proc. Natl. Acad. Sci. USA 90:2812-16 (1993); Huber B E et al., “Retroviral-mediated gene therapy for the treatment of hepatocellular carcinoma: An innovative approach for cancer therapy.” Proc. Natl. Acad. Sci. USA 88:8039-43 (1991); Hwu P et al., “Functional and Molecular Characterization of Tumor-Infiltrating Lymphocytes Transduced with Tumor Necrosis Factor-.alpha. cDNA for the Gene Therapy of Cancer in Humans.” J. Immunol. 150(9):4104-15 (1993); Kay M A et al., “Hepatic Gene Therapy: Persistent Expression of Human .alpha.1-Antitrypsin in Mice after Direct Gene Delivery In Vivo.” Hum. Gene Ther. 3:641-47 (1992); Lee N S et al., “Expression of small interfering RNAs targeted against hIV-1 rev transcripts in human cells.” Nature Biotechnol. 20:500-05 (2002); Lemarchand P et al., “Adenovirus-mediated transfer of a recombinant human .alpha..sub.1-antitrypsin cDNA to human endothelial cells.” Proc. Natl. Acad. Sci. USA 89:6482-86 (1992); Manoharan et al., “Chemical Modifications to Improve Uptake and Bioavailability of Antisense Oligonucleotides.” Ann. N.Y. Acad. Sci., 660:306-309 (1992); McLaughlin S K et al., “Adeno-Associated Virus General Transduction Vectors: Analysis of Proviral Structures.” J. Virol. 62(6):1963-73 (1988); Miller A D, “Progress Toward Human Gene Therapy.” Blood 76(2):271-78 (1990); Muzyczka N, “Use of Adeno-Associated Virus as a General Transduction Vector for Mammalian Cells.” Curr. Top. Microbiol. Immunol. 158:97-129 (1992); Quantin B et al., “Adenovirus as an expression vector in muscle cells in vivo.” Proc. Natl. Acad. Sci. USA 89:2581-84 (1992); Rosenfeld M A et al., “In Vivo Transfer of the Human Cystic Fibrosis Transmembrane Conductance Regulator Gene to the Airway Epithelium.” Cell 68:143-55 (1992); Rosenfeld M A et al., “Adenovirus-Mediated Transfer of a Recombinant .alpha.1-Antitrypsin Gene to the Lung Epithelium in Vivo.” Science 252:431-34 (1991); Samulski R J et al., “Helper-Free Stocks of Recombinant Adeno-Associated Viruses: Normal Integration Does Not Require Viral Gene Expression.” J. Virol. 63(9):3822-28 (1989); San H et al., “Safety and Short-Term Toxicity of a Novel Cationic Lipid Formulation for Human Gene Therapy.” Human Gene Therapy 4:781-88 (1993); Sui G et al., “A DNA vector-based RNAi technology to suppress gene expression in mammalian cells.” Proc. Natl. Acad. Sci. USA 99(8):5515-20 (2002); Tratschin J D et al., “Genetic Analysis of Adeno-Associated Virus: Properties of Deletion Mutants Constructed In Vitro and Evidence for an Adeno-Associated Virus Replication Function.” J. Virol. 51(3):611-619 (1984); Tratschin J D et al., “A Human Parvovirus, Adeno-Associated Virus, as a Eucaryotic Vector: Transient Expression and Encapsidation of the Procaryotic Gene for Chloramphenicol Acetyltransferase.” Mol. Cell. Biol. 4(10):2072-81 (1984); Tratschin J D et al., “Adeno-Associated Virus Vector for High-Frequency Integration, Expression, and Rescue of Genes in Mammalian Cells.” Mol. Cell. Biol. 5(11):3251-60 (1985); van Beusechem V W et al., “Long-term expression of human adenosine deaminase in rhesus monkeys transplanted with retrovirus-infected bone-marrow cells.” Proc. Natl. Acad. Sci. USA 89:7640-44 (1992); Wilson J M et al., “Hepatocyte-directed Gene Transfer in Vivo Leads to Transient Improvement of Hypercholesterolemia in Low Density Lipoprotein Receptor-deficient Rabbits.” J. Biol. Chem. 267(2):963-67 (1992); Wilson J M et al., “Retrovirus-mediated transduction of adult hepatocytes.” Proc. Natl. Acad. Sci. USA 85:3014-18 (1988); Wolff J A et al., “Direct Gene Transfer into Mouse Muscle in Vivo.” Science 247:1465-68 (1990); Wondisford F E et al., “Cloning of the Human Thyrotropin .beta.-Subunit Gene and Transient Expression of Biologically Active Human Thyrotropin after Gene Transfection.” Mol. Endocrinol. 2(1):32-39 (1988); Wu G Y and Wu C H, “Receptor-mediated Gene Delivery and Expression in Vivo.” J. Biol. Chem. 263(29):14621-14624 (1988); Anderson, W. F., “Human Gene Therapy,” Nature, 392:25-30 (Apr. 30, 1998); Nishikawa, M. et al., “Nonviral Vectors in the Millennium: Delivery Barriers in Gene Transfer,” Human Gene Therapy, 12:861-870 (2001); Rozenberg, Y. et al., “Alternative Gene Delivery,” S.T.P. Pharma Sciences,11:21-30 (2001); Balicki, D. et al., “Gene Therapy of Human Disease,” Medicine, 81:69-86 (2002); Orkin, S. H. and Motulsky, A. G., “Report and Recommendations of the Panel to Assess the NIH Investment in Research on Gene Therapy”, National Institutes of Health, 1-41 (Dec. 7, 1995); Verma, I. M. et al., “Gene Therapy-Promises, Problems and Prospects,” Nature, 389:239-242 (1997); Ross, G. et al., “Gene Therapy in the United States: A Five-Year Status Report,” Human Gene Therapy, 7:1781-1790 (1996); Marshall, E., “Gene Therapy's Growing Pains,” Science, 269:1050-1055 (1995); Gunzburg, W. H. and Salmons, B., “Virus Vector Design in Gene Therapy,” Mol. Med. Today 1(9) Abstract (December 1995); Goorecki, D. C., “Prospects and Problems of Gene Therapy: An Update,” Expert Opin. Emerging Drugs 6(2):187-198 (2001); Deonarain, M. P., “Ligand-Targeted Receptor-Mediated Vectors for Gene Delivery,” Exp. Opin. Ther. Patents 8(1):53-69 (January 1998); U.S. Pat. Nos. 3,687,808; 4,469,863; 4,476,301; 4,587,044; 4,605,735; 4,667,025; 4,762,779; 4,789,737; 4,816,567; 4,824,941; 4,828,979; 4,835,263; 4,845,205; 4,868,116; 4,876,335; 4,904,582; 4,948,882; 4,958,013; 4,980,286; 4,981,957; 5,013,830; 5,023,243; 5,034,506; 5,082,830; 5,109,124; 5,112,963; 5,118,800; 5,118,802; 5,130,302; 5,134,066; 5,138,045; 5,149,797; 5,166,315; 5,166,320; 5,175,273; 5,177,196; 5,185,444; 5,188,897; 5,194,599; 5,214,134; 5,214,136; 5,216,141; 5,218,105; 5,220,007; 5,223,409; 5,225,539; 5,235,033; 5,245,022; 5,254,469; 5,256,775; 5,258,506; 5,262,536; 5,264,423; 5,264,562; 5,264,564; 5,272,250; 5,276,019; 5,278,302; 5,286,717; 5,292,873; 5,317,098; 5,319,080; 5,321,131; 5,359,044; 5,366,878; 5,367,066; 5,371,241; 5,391,723; 5,393,878; 5,399,676; 5,403,711; 5,405,938; 5,405,939; 5,414,077; 5,416,203; 5,432,272; 5,434,257; 5,446,137; 5,451,463; 5,453,496; 5,455,233; 5,457,187; 5,459,255; 5,466,677; 5,466,786; 5,470,967; 5,476,925; 5,484,908; 5,486,603; 5,489,677; 5,491,133; 5,502,177; 5,510,475; 5,512,439; 5,512,667; 5,514,785; 5,519,126; 5,519,134; 5,525,465; 5,525,711; 5,527,899; 5,536,821; 5,539,082; 5,541,306; 5,541,307; 5,541,313; 5,545,730; 5,550,111; 5,552,538; 5,552,540; 5,561,225; 5,563,253; 5,565,332; 5,565,350; 5,565,552; 5,565,555; 5,567,810; 5,567,811; 5,571,799; 5,574,142; 5,576,427; 5,578,717; 5,578,718; 5,580,731; 5,585,481; 5,587,361; 5,587,371; 5,587,469; 5,591,584; 5,591,722; 5,594,121; 5,595,726; 5,596,086; 5,596,091; 5,597,696; 5,597,909; 5,599,923; 5,599,928; 5,602,240; 5,608,046; 5,610,289; 5,610,300; 5,614,617; 5,618,704; 5,623,065; 5,623,070; 5,625,050; 5,627,053; 5,633,360; 5,639,873; 5,645,985; 5,646,265; 5,646,269; 5,652,355; 5,652,356; 5,658,873; 5,663,312; 5,670,633; 5,672,697; 5,677,437; 5,677,439; 5,681,941; 5,688,941; 5,700,920; 5,700,922; 5,714,331; 5,719,262; 5,721,218; 5,733,743; 5,750,692; 5,763,588; 5,770,713; 5,792,608; 5,792,747; 5,830,653; 5,871,907; 6,005,096; 6,656,730; 7,195,916; US 2006/0019256, WO 86/01533; WO 87/02671; WO 89/02468; WO 89/05345; WO 89/07136; WO 90/02809; WO 91/17271; WO 92/01047; WO 92/07573; WO 92/09690; WO 92/15679; WO 92/18619; WO 92/20791; WO 93/01288; WO 93/07883; WO 93/24510; WO 94/02610, each of which is expressly incorporated herein by reference.

According to another embodiment, a live bacterial vector, preferably a tumor targeting bacteria, such as Salmonella typhimurium VNP20009 (TAPET), is used to deliver an RNA or DNA construct, or an expressed gene product, such as L-PGDS, in or in proximity to, the tumor. For example, see U.S. Pat. Nos. 7,514,089; 7,452,531; 7,354,592; 6,962,696; 6,923,972; 6,863,894; 6,685,935; 6,475,482; 6,447,784; 6,190,657; 6,080,849; 2009/0175829; 2009/0053186; 2009/0028842; 2005/0026866; 2004/0202663 each of which is expressly incorporated herein by reference. See also, Chen G, Wei D P, Jia L J, Tang B, Shu L, Zhang K, Xu Y, Gao J, Huang X F, Jiang W H, Hu Q G, Huang Y, Wu Q, Sun Z H, Zhang J F, Hua Z C, “Oral delivery of tumor-targeting Salmonella exhibits promising therapeutic efficacy and low toxicity” Cancer Science, 100(12):2437-2443, December 2009.

According to a further aspect of the invention, pharmacological agents which target DP1 and/or DP2 are provided for the treatment of lung cancer.

L-PGDS transgenic overexpressors are relatively resistant to carcinogen-induced lung tumor formation and/or require a higher dose of carcinogen to elicit the same number of tumors per mouse than control. L-PGDS knockout mice to have increased tumor multiplicity and a high sensitivity to carcinogen-induced lung tumor formation.

DP1/DP2 agonists may provide protection against carcinogen-induced lung tumors or result in decreased tumor progression.

L-PGDS effects are modulated by its enzymatic product PGD₂ and it is believed to be the DP1 receptor responsible for the control of tumor growth. It is however possible downstream PGD₂-derivatives, working via PPARγ interaction, are also involved. Studies using PPARγ agonists (15-dPGJ₂) as well as antagonists (LG100641) help confirm or rule out the involvement of signal transduction from PPARγ activation. Signaling studies permit defining a role for PKC, either acting alone or synergistically with p38MAPK, mediating L-PGDS-induced apoptosis. In addition, PI3-K may contribute to this process.

L-PGDS or synthetic receptor agonists (or other receptor-active drugs) for its enzymatic product prostaglandin D₂, have an enormous potential to positively impact lung tumor prevention/treatment. Given the relatively easy accessibility of the lung, L-PGDS gene therapy is also a viable option in the treatment of lung cancer.

What is claimed is:

1. A method of treating a non small cell lung cancer, comprising administering an effective amount of a Prostaglandin D₂ (PGD₂) receptor agonist, in a pharmaceutically acceptable form, to a patient having non small cell lung cancer, in sufficient quantity to treat the non small cell lung cancer.

2. The method according to claim 1, wherein the PGD₂ receptor agonist comprises at least one of BW245C and BW868C.

3. The method according to claim 1, further comprising performing an assay on the non small cell lung cancer cells to determine a Lipocalin-type prostaglandin D2 synthase (L-PGDS) activity or expression of the tissue, and administering the PGD₂ agonist selectively in dependence on a determined low level of L-PGDS in the non small cell lung cancer cells.

4. A method of diagnosing, staging or predicting outcome of a non small cell lung cancer tumor, comprising testing cells of the non small cell lung cancer tumor for at least one of indicia or mRNA level corresponding to the Lipocalin-type prostaglandin D synthase (L-PGDS) gene, L-DPGS gene product, and PGD₂ level, and scoring the test result with respect to non-cancer lung cells.

5. The method according to claim 4, wherein the at least one indicia or mRNA level corresponding to the Lipocalin-type prostaglandin D synthase (L-PGDS) gene, L-PGDS gene product, and PGD₂ level of the cells shows at least a 40% reduction as compared to non-cancer lung cells from the same patient.

6. The method according to claim 4, wherein the at least one indicia or mRNA level corresponding to the Lipocalin-type prostaglandin D synthase (L-PGDS) gene, L-PGDS gene product, and PGD₂ level of the cells shows at least a 60% reduction as compared to non-cancer lung cells from the same patient.

7. The method according to claim 4, wherein the at least one indicia or mRNA level corresponding to the Lipocalin-type prostaglandin D synthase (L-PGDS) gene, L-PGDS gene product, and PGD₂ level of the cells shows at least an 80% reduction as compared to non-cancer lung cells from the same patient.

8. The method according to claim 4, wherein an mRNA level of Lipocalin-type prostaglandin D synthase (L-PGDS) gene transcript of the tested cells of less than 40% of non-cancerous lung cells from the same patient indicates a poor prognosis if untreated.

9. The method according to claim 4, wherein an mRNA level of Lipocalin-type prostaglandin D synthase (L-PGDS) gene transcript of the tested cells of less than 40% of non-cancerous lung cells from the same patient indicates a likely effective response of the non small cell lung cancer tumor to a therapy which increases L-PGDS activity or agonizes PGD₂ receptors in the non small cell lung cancer tumor.

10. The method according to claim 4, further comprising reporting a deviation from normal at least one indicia or mRNA level corresponding to the Lipocalin-type prostaglandin D synthase (L-PGDS) gene, L-PGDS gene product, and PGD₂ level of the cells shows at least a 25% reduction as compared to non-cancer lung cells from the same patient.

11. A method of treating a non small cell lung cancer tumor in a patient, comprising testing cells from a biopsy of the non small cell lung cancer tumor for at least one of indicia or mRNA corresponding to the Lipocalin-type prostaglandin D synthase (L-PGDS) gene, L-PGDS gene product, and PGD₂ level, and comparing the biopsied non small cell lung cancer tumor cells with control lung cells, and treating the non small cell lung cancer tumor with a treatment to increase L-PGDS or agonize PGD₂ receptors in the non small cell lung cancer tumor selectively in dependence on the testing, wherein a reduced level of the at least one of indicia or mRNA corresponding to the Lipocalin-type prostaglandin D synthase (L-PGDS) gene, L-PGDS gene product, and PGD₂ level indicates a likely favorable response to the treatment.

12. A method of treating a patient having a non small cell lung cancer, comprising administering an effective amount of a gene therapy configured to cause expression in lung tissue of the patient of Lipocalin-type prostaglandin D synthase (L-PGDS).

13. The method according to claim 12, wherein the gene therapy comprises a genetically engineered adenovirus or SV40 virus comprising DNA encoding an L-PGDS.

14. The method according to claim 13, wherein the L-PGDS comprises a human L-PGDS EC=5.3.99.2 and the virus comprises an adenovirus.

15. The method according to claim 12, wherein the expressed L-PGDS has an activity in the lung tissue higher than normal human L-PGDS.

16. The method according to claim 12, wherein the gene therapy is applied intratracheally.

17. The method according to claim 12, further comprising administering a PGD₂ receptor agonist to the patient.

18. The method according to claim 16, wherein the PGD₂ receptor agonist comprises at least one of BW245C and BW868C.

19. A method to predict pathological characteristics of a non small cell lung cancer tumor in a patient, comprising:
performing an assay to determine expression of a gene encoding an L-PGDS in the non small cell lung cancer tumor and a non-tumor margin; and categorizing the pathological characteristics of the non small cell lung cancer tumor, selectively in dependence on the assay.

20. The method according to claim 19, further comprising treating the patient in accordance with the categorized pathological characteristics.
